(19) United States
(12) Patent Application Publication
(10) Pub. No.: US 2024/0306984 A1
(43) Pub. Date: Sep. 19, 2024

(54) DEVICES AND METHODS FOR APPLYING STIMULATION IN THE ASSESSMENT OF CONSCIOUSNESS

(71) Applicant: Dignity Health, San Francisco, CA (US)

(72) Inventors: Brandon Fox, San Francisco, CA (US); Dakota Graham, San Francisco, CA (US)

(21) Appl. No.: 18/576,509

(22) PCT Filed: Jul. 11, 2022
(86) PCT No.: PCT/US2022/036735

§ 371 Date: Jan. 4, 2024

Related U.S. Application Data
(60) Provisional application No. 63/231,064, filed on Aug. 9, 2021.
(60) Provisional application No. 63/219,877, filed on Jul. 9, 2021.

(51) Int. Cl. A61B 5/00; A61B 90/00

(52) U.S. Cl. CPC A61B 5/483; A61B 5/6826; A61B 5/6838; A61B 5/742; A61B 90/06; A61B 2090/064; A61B 2560/0431; A61B 2560/0468

(57) ABSTRACT

A device applies standardized central and peripheral stimuli to a patient for assessment of consciousness. The device is hand-held and includes a first stimulus element for application of peripheral stimuli to a digit (e.g., a finger or a toe) of the patient and a second stimulus element for application of central stimuli to the patient, enabling practitioners to rapidly switch between peripheral and central stimulus application. The device can generate a standardized electrical stimulus, thermal stimulus, and/or mechanical stimulus for application for improved accuracy when assessing consciousness of the patient by methods such as the Glasgow Coma Scale. In another aspect, the device applies standardized stimuli at an appropriate intensity to elicit a response without causing tissue damage.

### CROSS-REFERENCE TO RELATED APPLICATIONS

This is a PCT application that claims benefit to U.S. Provisional Patent Application Ser. Nos. 63/219,877 and 63/231,064 filed Jul. 9, 2021 and Aug. 9, 2021, respectively, which are herein incorporated by reference in their entireties.

### FIELD

The present disclosure generally relates to assessment of a status of a patient and in particular, to devices and associated methods for applying peripheral and/or central stimulation in the assessment of consciousness to elicit a response from the patient.

### BACKGROUND

Current methods of applying stimuli to assess consciousness in critically ill and/or injured patients include the Glasgow Coma Scale in which a practitioner applies painful stimuli to one or more areas of a patient's body and observes their response. This typically involves application of painful central and/or peripheral stimuli by methods including trapezius squeeze, earlobe pinch, supraorbital pressure, sternal rub, and nail-bed pressure. Because these methods are normally manually applied by practitioners, the intensity of the stimuli can be inconsistent and sometimes can require repeated attempts which can adversely affect patient outcomes. Moreover, continued re-assessment can result in injury to the patient such as bruising and continued pain, with some methods being described as “barbaric”. This can obviously be quite upsetting to the patient, their loved ones, and the practitioners themselves. As such, it is necessary to develop a safer and more reproducible method to apply noxious stimuli during assessment of patients with depressed level of consciousness to reduce unnecessary care, delays in care, and patient injury.

It is with these observations in mind, among others, that various aspects of the present disclosure were conceived and developed.

### BRIEF DESCRIPTION OF THE DRAWINGS

FIGS. 1A-1D are a series of illustrations showing a device for applying stimulation in assessment of consciousness applying a peripheral stimulus to a patient, where FIGS. 1A-1C show application of a peripheral stimulus to a finger of the patient and FIG. 1D shows application of a peripheral stimulus to a toe of the patient;

FIGS. 1E and 1F are a series of illustrations showing the device of FIGS. 1A-1D applying a central stimulus to a surface of the patient, particularly the supraorbital notch along the forehead;

FIGS. 2A and 2B are a pair of illustrations showing cross-sectional views of the device of FIGS. 1A-1F;

FIGS. 2C-2E are a series of simplified schematic diagrams showing various electronic components of the device of FIGS. 1A-1F;

FIGS. 3A-3C are a series of illustrations showing an electrical embodiment of the device of FIGS. 1A-1F in a first closed configuration and a second open configuration;

FIG. 3D is an illustration showing the device of FIGS. 3A-3C in an exploded configuration;

FIGS. 4A and 4B are a pair of simplified illustrations showing a first electrode configuration and a second electrode configuration of the device of FIGS. 3A-3C;

FIGS. 4C and 4D are a pair of simplified schematic diagrams showing various electronic components of the device of FIGS. 3A-3D;

FIG. 5A is an illustration showing the device of FIGS. 3A-3C with a first body portion removed;

FIG. 5B is an illustration showing a cross-sectional view of the device of FIGS. 3A-3C;

FIGS. 6A-6F are a series of simplified illustrations showing various electrode configurations of the device of FIGS. 3A-3C;

FIG. 6G is an illustration showing the device of FIGS. 3A-3C with the electrode configuration of FIGS. 6C and 6D;

FIG. 7A is an illustration showing a thermal embodiment of the device of FIGS. 1A-1F in a first closed configuration;

FIGS. 7B and 7C are a pair of illustrations showing a proximal portion and a distal portion of the device of FIG. 7A;

FIGS. 8A and 8B are a pair of illustrations showing the device of FIGS. 7A-7C in a first closed configuration and a second closed configuration;

FIGS. 8C and 8D are a pair of illustrations showing cross-sectional views of the device of FIGS. 8A and 8B in a first closed configuration and a second closed configuration;

FIGS. 9A and 9B are a pair of illustrations showing a simplified view of a switching component of the device of FIGS. 7A-7C;

FIG. 9C is a simplified schematic diagram showing various electronic components of the device of FIGS. 7A-7B;

FIG. 10 is an illustration showing a mechanical embodiment of the device of FIGS. 1A-1F;

FIGS. 11A and 11B are a pair of illustrations showing cross-sectional views of the device of FIG. 10 in a first closed configuration and a second closed configuration, where the device of FIG. 10 includes an electronically assisted monitoring component;

FIG. 11C is an illustration showing a digital indicator of the device of FIG. 10;

FIG. 11D is a simplified schematic diagram showing various electronic components of the device of FIGS. 11A-11C;

FIGS. 12A and 12B are a pair of illustrations showing cross-sectional views of the device of FIG. 10 in a first closed configuration and a second closed configuration, where the device of FIG. 10 includes a purely mechanical monitoring component;

FIG. 12C is an illustration showing an analog indicator of the device of FIGS. 12A and 12B;

FIGS. 13A and 13B are process flows showing a method of applying peripheral stimuli to a patient using the devices of FIGS. 1A-12C; and

FIGS. 14A and 14B are process flows showing a method of applying central stimuli to a patient using the devices of FIGS. 1A-12C.

Corresponding reference characters indicate corresponding elements among the view of the drawings. The headings used in the figures do not limit the scope of the claims.

### DETAILED DESCRIPTION

A consciousness assessment stimulus device applies central and peripheral stimuli to a patient to elicit a physical reaction from the patient for assessment of consciousness. Previous methods of applying painful central and/or peripheral stimuli by methods include trapezius squeeze, earlobe pinch, supraorbital pressure, sternal rub, and nail-bed pressure, which can be inconsistent each time the stimuli is applied resulting in inaccurate evaluation and potential injury to the patient. Further, some cases, it can be necessary to rapidly switch between application of central and peripheral stimulation. Additional problems can arise when patients involuntarily pull away from stimuli, making it difficult for practitioners to maintain consistent application for accurate assessment.

As such, the present disclosure provides devices and associated methods for applying standardized central and peripheral stimuli to a patient for assessment of consciousness. The device is hand-held and includes a first stimulus element for application of peripheral stimuli to a digit (e.g., a finger or a toe) of the patient and a second stimulus element for application of central stimuli to the patient, enabling practitioners to rapidly switch between peripheral and central stimulus application. In a preferred embodiment, the device captures a digit of the patient for application of peripheral stimuli and applies a clamping force to the digit to enable the practitioner to maintain their grasp on the digit for continued application if/when the patient involuntarily pulls away. The device can generate a standardized electrical stimulus, thermal stimulus, and/or mechanical stimulus for application to the patient that is consistent each time the stimulus is applied for improved accuracy when assessing consciousness of the patient. In another aspect, the device applies standardized stimuli at an appropriate intensity to elicit a response without causing tissue damage.

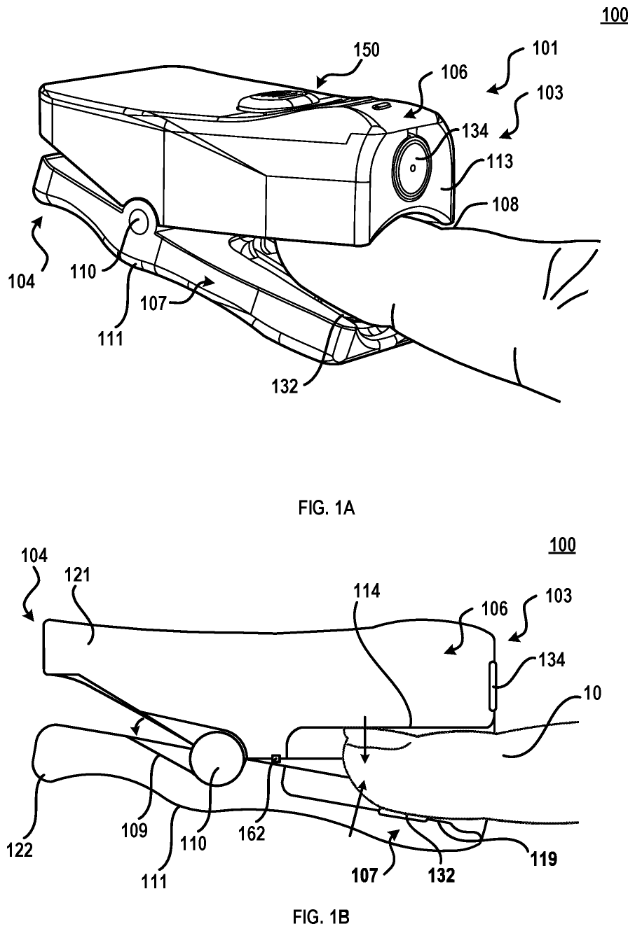

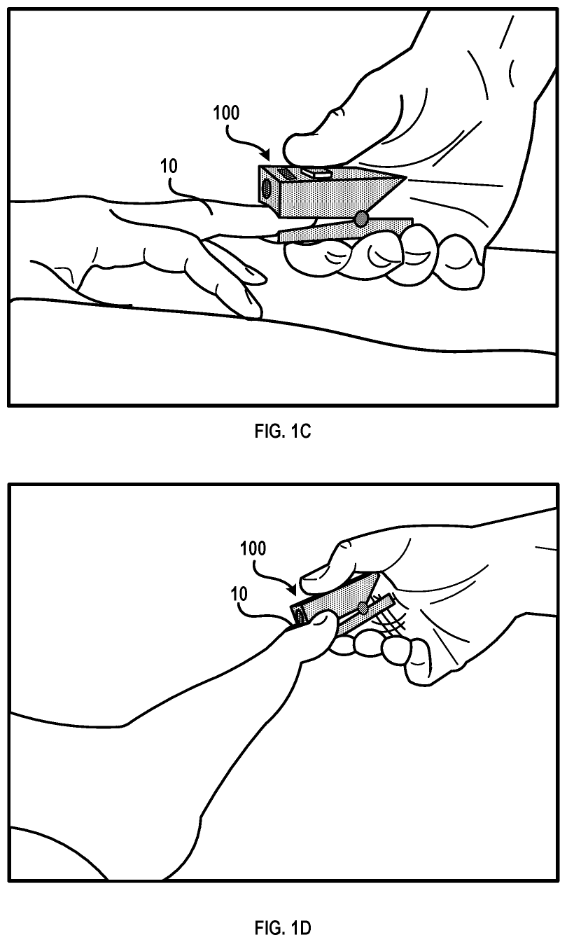

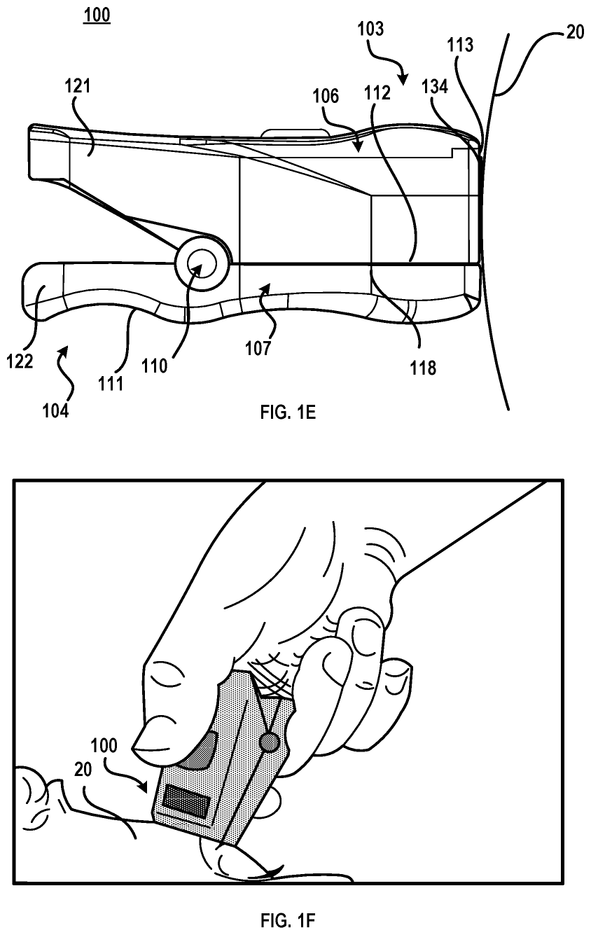

With reference to FIGS. 1A-1F, a device 100 is operable for generating and applying a stimulus to a patient, including an electrical stimulus, a thermal stimulus, and/or a mechanical stimulus. The device includes a housing 101 defining an internal cavity 105 that houses various electronic components of the device 100, including a stimulus generator device 130 in communication with a first stimulus element 132 for application of peripheral stimuli to a digit 10 of a patient and a second stimulus element 134 for application of central stimuli to a surface 20 of the patient. In particular, FIGS. 1A-1D show peripheral stimulation as applied to a digit 10 of the patient, where the digit 10 is a finger (FIGS. 1A, 1B and 1C) or a toe (FIG. 1D). FIGS. 1E and 1F show central stimulation as applied to a surface 20 of the patient, which can include the supraorbital notch. As will be further discussed herein, the first stimulus element 132 and the second stimulus element 134 can be operable for application of electrical, thermal, and/or mechanical stimuli to cause depolarization of nociceptive nerve fibers along the digit 10 or the surface 20 of the patient. In some embodiments, the device 100 can be configured to apply more than one type of stimuli (electrical, thermal and/or mechanical).

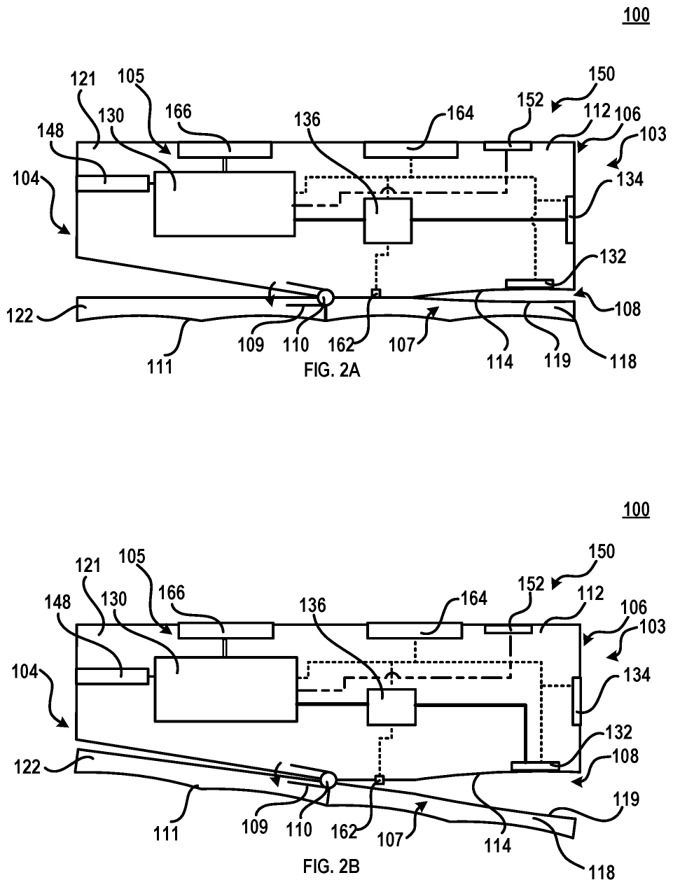

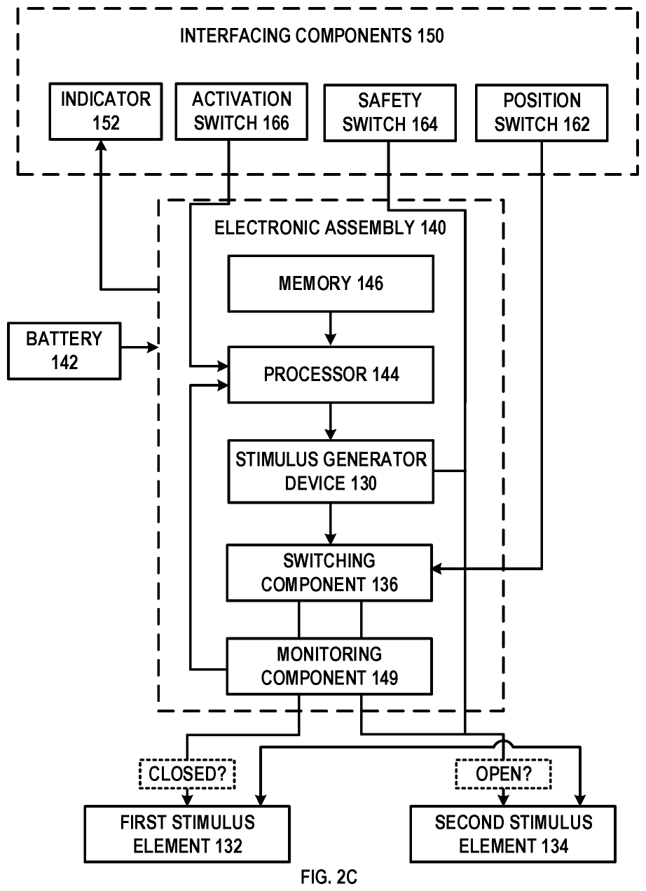

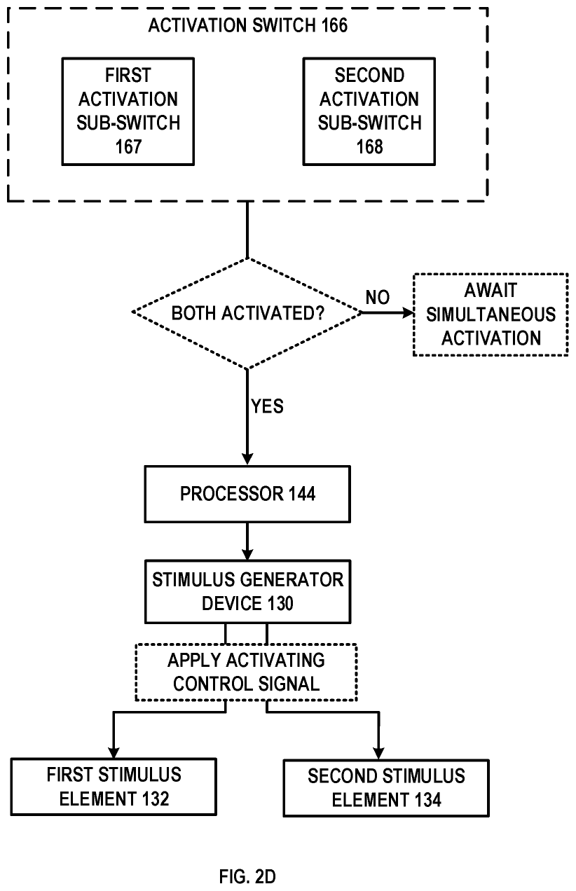

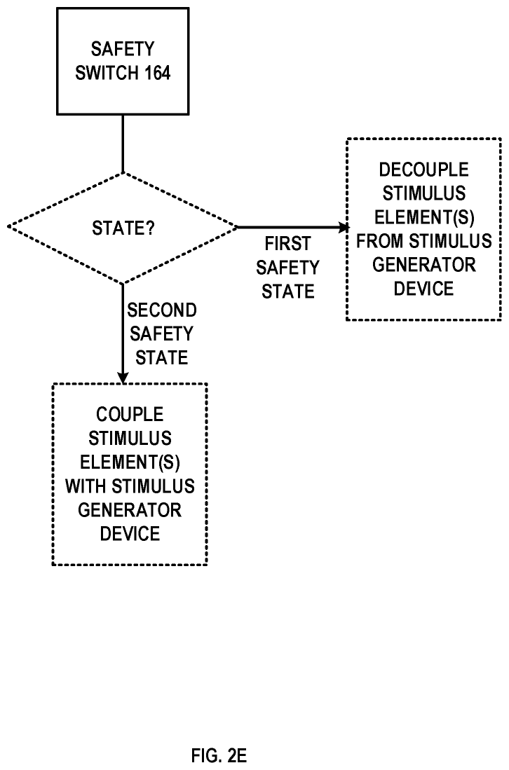

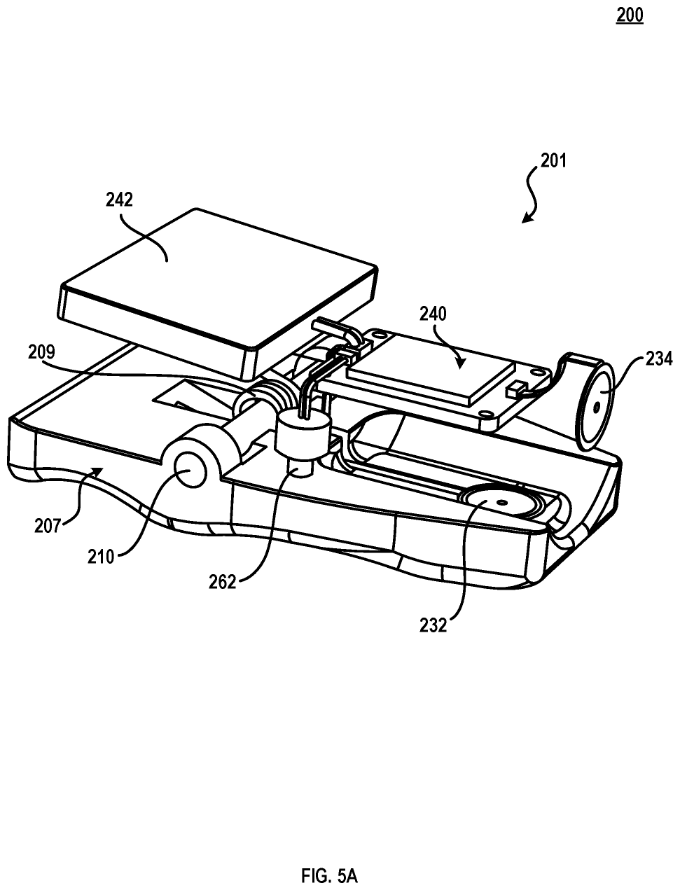

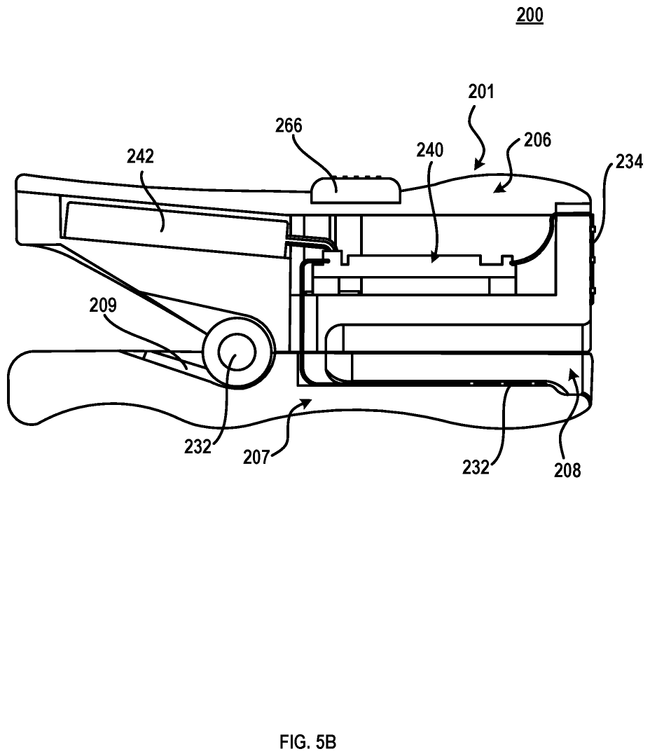

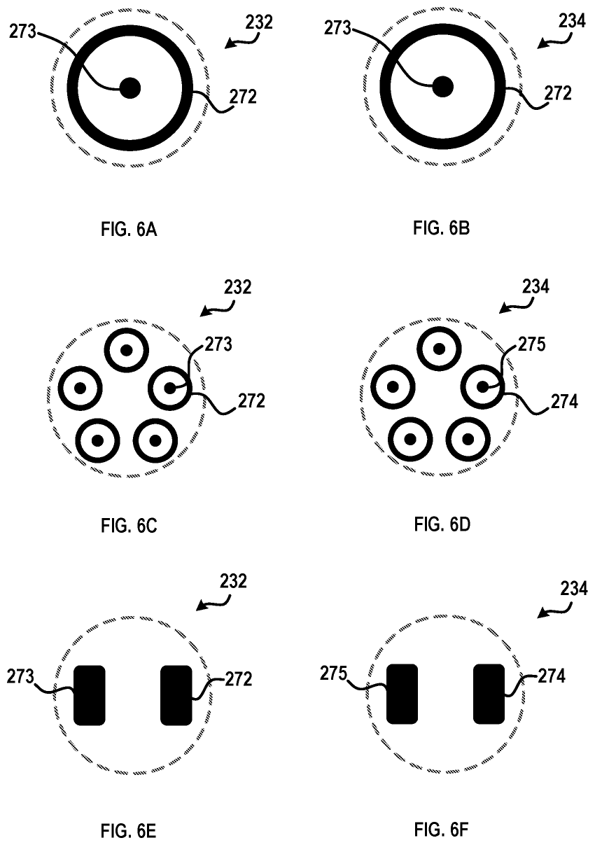

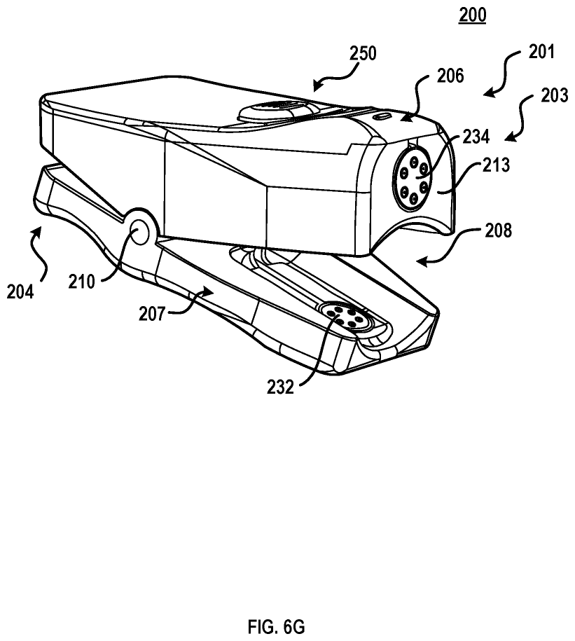

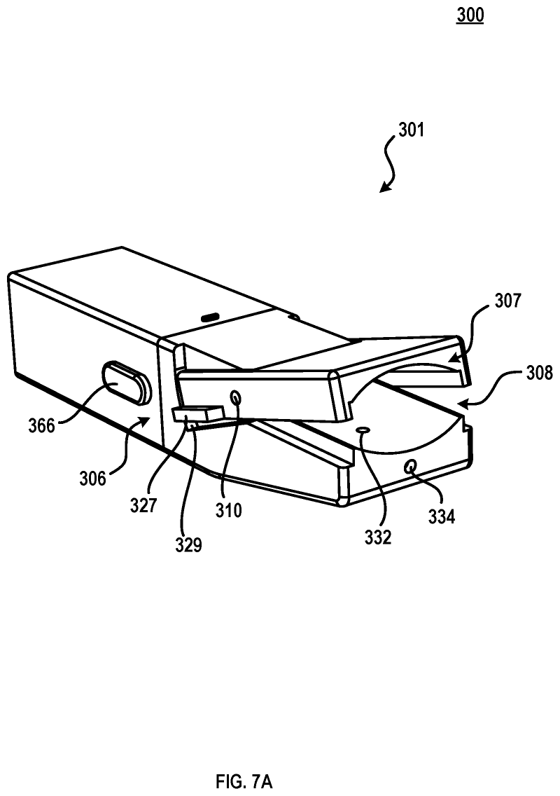

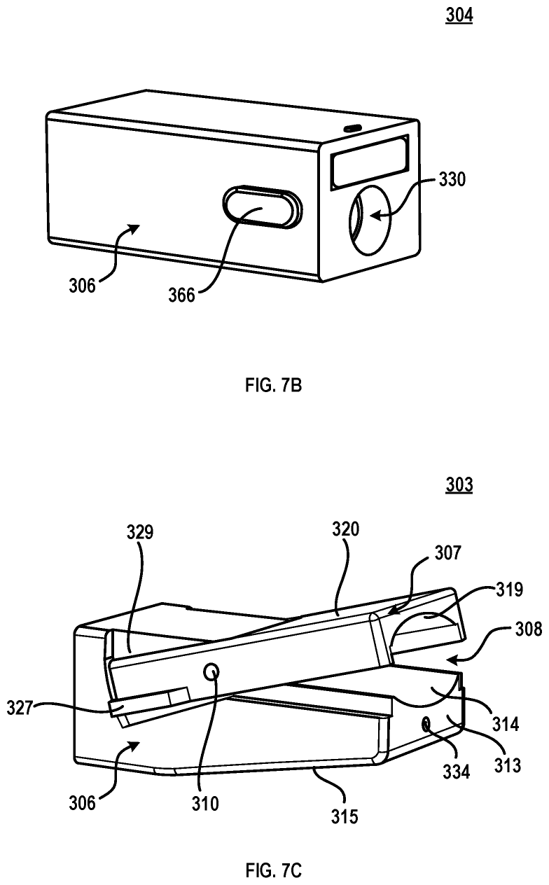

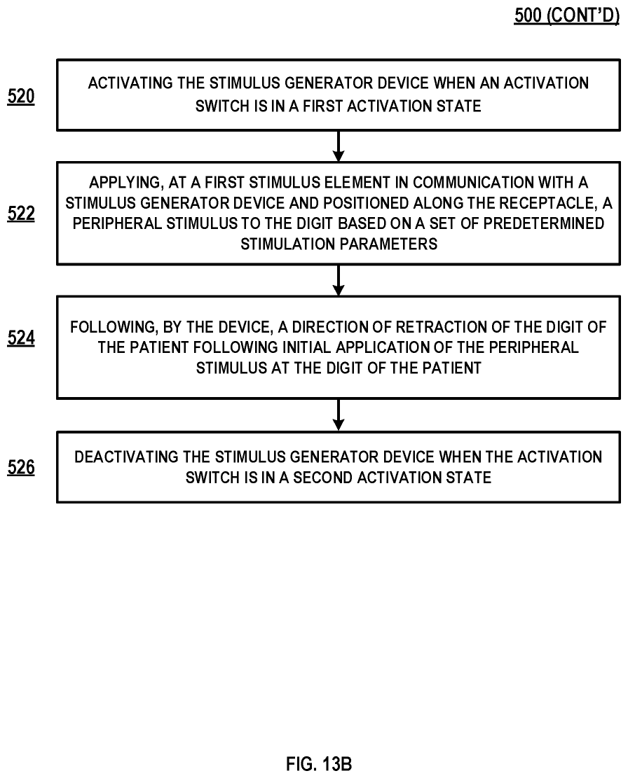

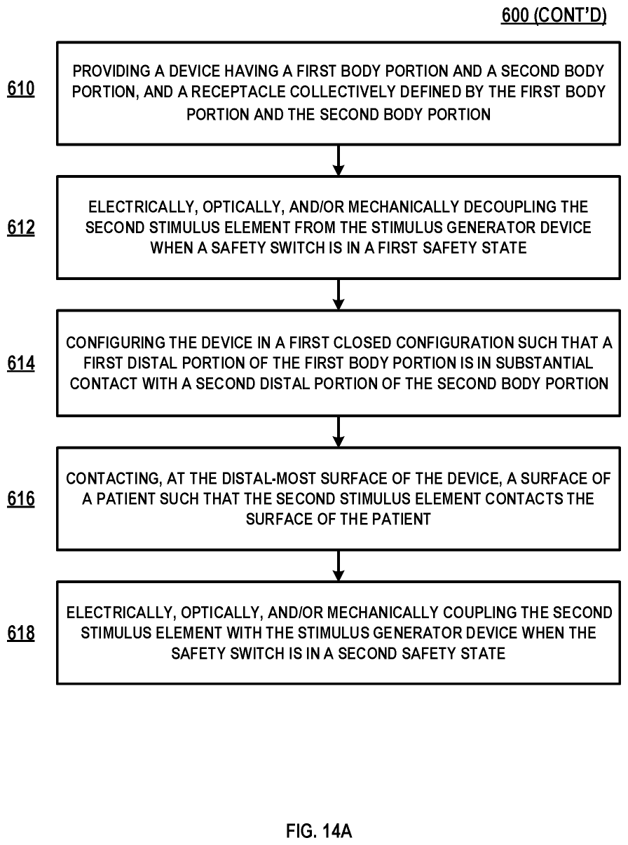

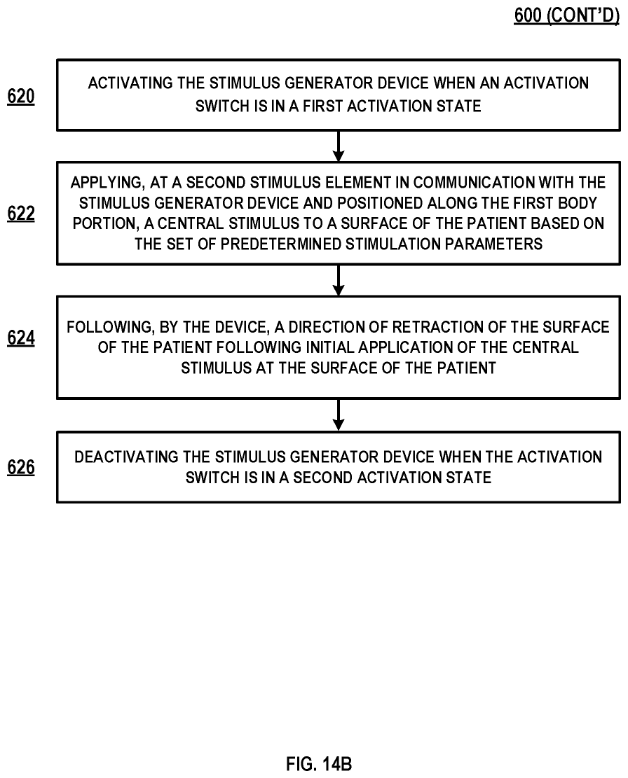

FIGS. 1A-2D show a first genericized embodiment for application of peripheral stimuli and central stimuli to a patient by device 100. FIGS. 5A-6G show a second, more specific embodiment for application of peripheral stimuli and central stimuli in the form of an electrical stimulus to a patient by device 200. FIGS. 7A-9C show a third embodiment for application of peripheral stimuli and central stimuli in the form of a thermal stimulus to a patient by device 300. FIGS. 10-12C show a fourth embodiment for application of peripheral stimuli and central stimuli in the form of a mechanical stimulus to a patient by device 400. FIGS. 13A-14B show methods 500 and 600 for applying peripheral and central stimuli to a patient using the device 100 (or 200, 300, or 400).

### Device Body

With continued reference to FIGS. 1A-1F, and with additional reference to FIGS. 2A-2E, the housing 101 defines a distal portion 103 and a proximal portion 104 defined opposite from the distal portion 103. A practitioner grasps, opens/closes, and operates the device 100 at the proximal portion 104 of the device and the distal portion 103 interacts with the patient when applying the stimulus. In some embodiments, the proximal portion 104 includes one or more interfacing components 150 including displays, switches and/or buttons that enable the practitioner to operate the device 100, and can further include one or more gripping portions 111 to improve a practitioner's grasp and control over the device 100. The housing 101 can include a first body portion 106 and a second body portion 107 linked by a biasing element 109 and a hinge 110 such that the first body portion 106 and the second body portion 107 collectively form a clamp or clip mechanism that is operable for assuming a first “closed” configuration (FIG. 2A) and a second “open” configuration (FIG. 2B). In the first closed configuration, the device 100 can apply a central stimulus to the surface 20 of the patient as shown in FIGS. 1E and 1F. Conversely, in the second open configuration, the device 100 can apply a peripheral stimulus to the digit 10 of the patient as shown in FIGS. 1A-1D. As shown, the first body portion 106 and the second body portion 107 collectively form a receptacle 108 at the distal portion 103 of the housing 101 that captures the digit 10 of the patient for application of a peripheral stimulus. The receptacle 108 includes the first stimulus element 132 that contacts the digit 10 of the patient to apply the peripheral stimulus.

The first body portion 106 defines a first distal portion 112 and the second body portion 107 defines a second distal portion 118 that collectively form the distal portion 103 of the housing 101. The first distal portion 112 can include a first inward-facing surface 114 that forms a part of the receptacle 108 and contacts the digit 10 of the patient when the digit 10 is captured at the receptacle 108; similarly, the second distal portion 118 can include a second inward-facing surface 119 that forms a part of the receptacle 108 and contacts the digit 10 of the patient when the digit 10 is captured at the receptacle 108. The first stimulus element 132 can be positioned along either the first inward-facing surface 114 as shown in the examples of FIGS. 2A and 2B or the second inward-facing surface 119 as shown in the examples of FIGS. 1A and 1B. The biasing element 109 biases the first body portion 106 and the second body portion 107 towards the first closed configuration such that the first distal portion 112 of the first body portion 106 is in substantial contact with the second distal portion 118 of the second body portion 107.

Similarly, the first body portion 106 defines a first proximal portion 121 and the second body portion 107 defines a second proximal portion 122 that collectively form the proximal portion 104 of the housing 101. The first proximal portion 121 and/or the second proximal portion 122 can include the one or more gripping portions 111 to further aid a practitioner in grasping and operating the device 100. In some embodiments, the first body portion 106 can include the one or more interfacing components 150 that enable a practitioner to operate the device 100.

In some embodiments, a practitioner can transition the device 100 from the first closed configuration to the second open configuration by pinching the first body portion 106 and the second body portion 107 together at the proximal portion 104 of the device 100. In the “open” configuration, the first distal portion 112 of the first body portion 106 and the second distal portion 118 of the second body portion 107 are forced apart as shown in the examples of FIGS. 1A, 1B and 2A. Similarly, a practitioner can transition the device 100 from the second open configuration to the first closed configuration by releasing the first body portion 106 and the second body portion 107 together at the proximal portion 104 of the device 100 as shown in the examples of FIGS. 1E and 2B.

As shown in FIGS. 1A-1D, to apply a peripheral stimulus to the digit 10 of a patient, the practitioner can transition the device 100 to the “open” configuration and insert the digit 10 into the receptacle 108 such that the device 100 remains in the “open” configuration while the digit 10 is present within the receptacle 108. The biasing element 109, being biased towards the closed configuration, generates a clamping force applied to the digit 10 when the first body portion 106 and the second body portion 107 are in the second open configuration to capture the digit 10 within the receptacle 108 and keep the digit 10 in contact with the first stimulus element 132 for continued application of the peripheral stimulus if/when the patient involuntarily pulls away. A practitioner can activate the first stimulus element 132 by pressing an activation switch 166 in association with the first stimulus element 132; in some embodiments, the device 100 can include a position switch 162 that is operable to prevent the first stimulus element 132 from activating unless the device 100 is in the “open” configuration.

The first body portion 106 defines a distal-most surface 113 including the second stimulus element 134 that contacts a surface 20 of the patient to apply the central stimulus. As shown in FIGS. 1E and 1F, to apply a central stimulus to the surface 20 of a patient, the practitioner can transition the device 100 to the “closed” configuration and contact the surface 20 with the second stimulus element 134. The practitioner can activate the second stimulus element 134 by pressing the activation switch 166 in association with the second stimulus element 134; in some embodiments, the position switch 162 can be operable to prevent the second stimulus element 134 from activating unless the device 100 is in the “closed” configuration to prevent unintentional activation.

In a primary embodiment, the first body portion 106 can be larger than the second body portion 107 and can include the internal cavity 105 that houses an electronic assembly 140 of the device 100 including the stimulus generator device 130.

### Electronic Assembly

As discussed above and with additional reference to FIGS. 2C and 2E, the device 100 includes the first stimulus element 132 and the second stimulus element 134 that respectively apply peripheral and central stimuli to the patient according to a set of predetermined stimulation parameters when the first stimulus element 132 or the second stimulus element 134 is in contact with the patient. In some embodiments, the first stimulus element 132 and the second stimulus element 134 are operable to apply an electrical stimulus to the patient when in contact with the patient in the form of an alternating voltage and/or current according to the set of predetermined stimulation parameters. In other embodiments, the first stimulus element 132 and the second stimulus element 134 are operable to apply a thermal stimulus to the patient in the form of a laser having an output wavelength according to the set of predetermined stimulation parameters that results in a stimulating temperature. In a further embodiment, the first stimulus element 132 and the second stimulus element 134 are operable to apply a mechanical stimulus to the patient in the form of a mechanical force having a force value according to the set of predetermined stimulation parameters. In one aspect, the peripheral and central stimuli applied through the first stimulus element 132 and the second stimulus element 134 cause depolarization of nociceptive nerve fibers in the patient.

The electronic assembly 140 of the device 100 supplies power and/or control signals that enable the first stimulus element 132 and the second stimulus element 134 to apply the peripheral and central stimuli to the patient and also include various safety mechanisms to ensure safe operation of the device 100. The electronic assembly 140 is included within the internal cavity 105 of the housing 101 of the device 100, which can in some embodiments be within the first body portion 106 of the device 100.

In particular, the electronic assembly 140 includes a battery 142 that provides power to the rest of the electronic assembly 140 for stimulus application and control. The battery 142 can receive power through a charging port 148 positioned along the housing 101 of the device 100.

Further, the electronic assembly 140 includes a processor 144 that manages control signals for the device 100, including various safety mechanisms. The processor 144 can be positioned within the internal cavity 105 of the housing 101. The processor 144 communicates with the one or more interfacing components 150 of the device 100 to receive control inputs from the practitioner and to provide indicators to the practitioner when operating the device 100. The electronic assembly 140 can also include a memory 146 that communicates with the processor 144 and includes instructions that dictate operating parameters of the device 100, including a set of predetermined stimulation parameters which can include maximum and/or minimum voltages to be applied to the first stimulus element 132 and the second stimulus element 134 and various fail-safe measures. For instance, the memory 146 can include instructions that cause the processor 144 to require simultaneous activation of more than one control input from the one or more interfacing components 150 of the device 100 in order to activate the first stimulus element 132 or the second stimulus element 134, and can also include instructions that allow only one of the first stimulus element 132 or the second stimulus element 134 to activate at a time as discussed above. The electronic assembly 140 can also include a monitoring component 149 that monitors the stimulus being applied at the first stimulus element 132 or the second stimulus element 134 to ensure proper operation of the device 100.

The one or more interfacing components 150 of the device 100 can be positioned along the housing 101 and can provide control inputs to the processor 144 and display indicator outputs from the processor 144 that convey operating information about the device 100. The one or more interfacing components 150 can include the activation switch 166 that provide an activation control input to the processor 144 when activated (e.g., when pressed by the practitioner). The activation switch 166 can be in the form of a switch or a button, and in some embodiments can include more than one sub-button. For instance, as shown in FIG. 2D, the activation switch 166 can include a first activation sub-button 167 and a second activation sub-button 168, and the instructions within the memory 146 can cause the processor 144 to activate the first stimulus element 132 or the second stimulus element 134 when both the first activation sub-button 167 and the second activation sub-button 168 are pressed or otherwise activated simultaneously. The processor 144 can apply an activating control signal to the stimulus generator device 130 to apply the stimulus at the first stimulus element 132 or the second stimulus element 134 upon sufficient activation of the activation switch 166. Further, in some embodiments, the one or more interfacing components 150 can include an intensity modulation component (not shown) that modulates an intensity of the stimulus being applied at the first stimulus element 132 or the second stimulus element 134.

The processor 144 can communicate with the stimulus generator device 130 to apply and control the stimulus being generated at the first stimulus element 132 or the second stimulus element 134. The stimulus generator device 130 can vary between the type of stimulus that is being applied. For instance, to apply an electrical stimulus, the stimulus generator device 130 can include a waveform generator. To apply a thermal stimulus, the stimulus generator device 130 can include a laser generator where the laser results in an applied temperature at the patient. In a primary embodiment, the processor 144 can apply an activating control signal to the stimulus generator device 130 upon receipt of an activation control input from the activation switch 166.

With reference to FIGS. 2C and 2E, the one or more interfacing components 150 can further include a safety switch 164 that, when activated, prevents the activating control signals from reaching one or more of the stimulus generator device 130, the first stimulus element 132 or the second stimulus element 134. As such, the safety switch 164 is operable for assuming a first safety state and a second safety state, where the safety switch 164 is operable for electrically, optically, and/or mechanically decoupling the first stimulus element 132 and/or the second stimulus element 134 from the stimulus generator device 130 when the safety switch is in the first safety state and for electrically, optically, and/or mechanically coupling the first stimulus element and/or the second stimulus element with the stimulus generator device when the safety switch 164 is in the second safety state. The safety switch 164 can be in the form of a switch or a button. In some embodiments, the safety switch 164 can include a software-based component in the form of instructions stored within the memory 146 that require that the safety switch 164 be engaged or disengaged in order to enable the processor 144 to activate the first stimulus element 132 or the second stimulus element 134. For instance, if a practitioner activates the activation switch 166 but does not disengage the safety switch 164, then the processor 144 will not apply an activating control signal to the stimulus generator device 130. Further, in some embodiments, the safety switch 164 can provide a “break” between the processor 144, the activation switch 166 and/or the stimulus generator device 130 and the first stimulus element 132 or the second stimulus element 134 by electrically, optically, and/or mechanically decoupling an electrical, optical and/or mechanical connection between the processor 144 and/or the stimulus generator device 130 and the first stimulus element 132 or the second stimulus element 134 when the safety switch 164 is engaged.

In some embodiments, the one or more interfacing components 150 can also include the position switch 162 that dictates which of the first stimulus element 132 or the second stimulus element 134 is activated. In some embodiments, it is ideal for only one of the first stimulus element 132 or the second stimulus element 134 to be activated at a time to prevent accidental discharge or stimulus application; as such, the position switch 162 can control which one of the first stimulus element 132 or the second stimulus element 134 is activated. As shown, the position switch 162 can be positioned along the first inward-facing surface 114 of the first body portion 106 or the second inward-facing surface 119 of the second body portion 107, as such, the position switch 162 assumes a first positional state when the device 100 is in the first “closed” configuration and assumes a second positional state when the device 100 is in the second “open” configuration. In some embodiments, the position switch 162 can communicate with a switching component 136 that electrically, optically, or mechanically controls which of the first stimulus element 132 or the second stimulus element 134 is activated.

In a primary embodiment, when the device 100 is in the “closed” configuration (e.g., if a digit is not captured at the receptacle 108), then the position switch 162 and/or the switching component 136 can allow the second stimulus element 134 to activate when the activation switch 166 is activated but prevent the first stimulus element 132 from activating. When the device 100 is in the “open” configuration (e.g., if a digit is captured at the receptacle 108), then the position switch 162 and/or the switching component 136 can allow the first stimulus element 132 to activate when the activation switch 166 is activated but prevent the second stimulus element 134 from activating. The mechanism employed by the switching component 136 can vary based on the type of stimulus being applied by the device 100. For instance, for an electrical embodiment, the switching component 136 can be electrical in nature and can communicate directly with the processor 144 and/or the stimulus generator device 130 (which can include a waveform generator), and can include digital and/or analog circuitry to electrically decouple the first stimulus element 132 or the second stimulus element 134 (in electrode form) from the stimulus generator device 130. Similarly, in a mechanical embodiment in which the mechanical stimulus is a mechanical force controlled electronically, the switching component 136 can similarly be electrical in nature and can communicate directly with the processor 144 and/or the stimulus generator device 130 (which can include a force generator), and can include circuitry to electrically decouple the first stimulus element 132 or the second stimulus element 134 (in force applicator form) from the stimulus generator device 130. However, in other embodiments, the position switch 162 and/or the switching component 136 can be mechanical in nature (e.g., not directly connected to the processor 144).

The one or more interfacing components 150 can include one or more indicators 152 that provide information to a practitioner about operation of the device 100. The one or more indicators 152 can include a first status indicator that displays an on/off/error state of the device 100. Further, in some embodiments, the one or more indicators 152 can include a positional indicator that displays information related to which of the first stimulus element 132 or the second stimulus element 134 are being activated. In another aspect, the one or more indicators 152 can include an intensity indicator that displays information related to an intensity of stimulus being applied. Further, in some embodiments, the one or more indicators 152 can include a battery life indicator that provides information related to a battery life of the device 100 (e.g., charging, fully charged, low battery, etc.). In other embodiments, the one or more indicators 152 can be purely mechanical in nature.

To apply a peripheral stimulus, a practitioner can open the device 100 to the second open configuration and insert the digit 10 into the receptacle 108 such that the first stimulus element 132 makes substantial contact with the digit 10 and the clamping force generated by the biasing element 109 is applied at the digit 10. By transitioning the device 100 into the second open configuration, the position switch 162 and switching component 136 ensure that only the first stimulus element 132 can activate. If applicable, the practitioner can transition the safety switch 164 into the second safety state (e.g., “safety off”) to enable activation of the first stimulus element 132. The practitioner can then activate the activation switch 166 to activate the stimulus generator device 130 and apply the peripheral stimulus to the digit 10 according to the set of predetermined stimulation parameters stored within the memory 146. The monitoring component 149 can monitor the intensity of the applied stimulus and indicate operating data to the practitioner. It is expected for patients to involuntarily retract their digit 10 upon application of the peripheral stimulus; the device 100 enables the practitioner to maintain their grasp on the digit 10 and ensure effective application of the peripheral stimulus by following, with the device 100, a direction of retraction of the digit 10. Following successful application of the peripheral stimulus, the practitioner can release the activation switch 166 to stop the applied stimulus, and can return the safety switch 164 to the first safety state (e.g., “safety on”).

To apply a central stimulus, a practitioner can close the device 100 to the first closed configuration and contact the surface 20 of the patient with the distal-most surface 113 of the device 100 such that the second stimulus element 134 makes substantial contact with the surface 20. By transitioning the device 100 into the first closed configuration, the position switch 162 and switching component 136 ensure that only the second stimulus element 134 can activate. If applicable, the practitioner can transition the safety switch 164 into the second safety state (e.g., “safety off”) to enable activation of the second stimulus element 134. The practitioner can then activate the activation switch 166 to activate the stimulus generator device 130 and apply the central stimulus to the surface 20 according to the set of predetermined stimulation parameters stored within the memory 146. The monitoring component 149 can monitor the intensity of the applied stimulus and indicate operating data to the practitioner. It is expected for patients to involuntarily retract upon application of the central stimulus; the device 100 enables the practitioner to maintain their contact with the surface 20 and ensure effective application of the central stimulus by following, with the device 100, a direction of retraction of the surface 20. Following successful application of the central stimulus, the practitioner can release the activation switch 166 to stop the applied stimulus, and can return the safety switch 164 to the first safety state (e.g., “safety on”).

The handheld profile of the device 100 and automatic nature of the position switch 162 enables practitioners to rapidly and safely switch between peripheral and central stimulus application in practice. Further, by applying the stimuli according to the set of predetermined stimulation parameters and allowing the practitioner to monitor operating data, the device 100 enables consistent application of the same stimuli each time the device 100 is used, improving the efficacy of assessment methods such as the Glasgow Coma scale.

### Electrical Stimulus

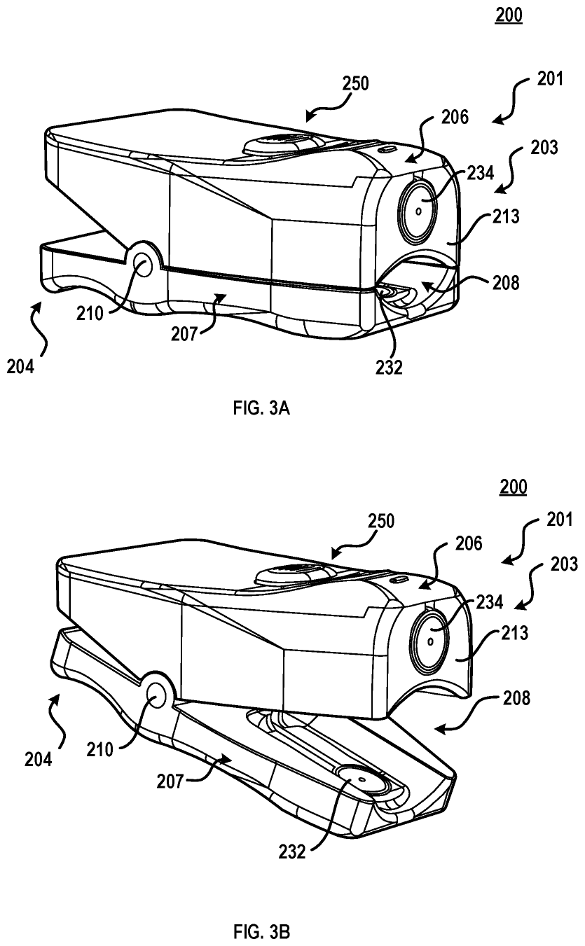

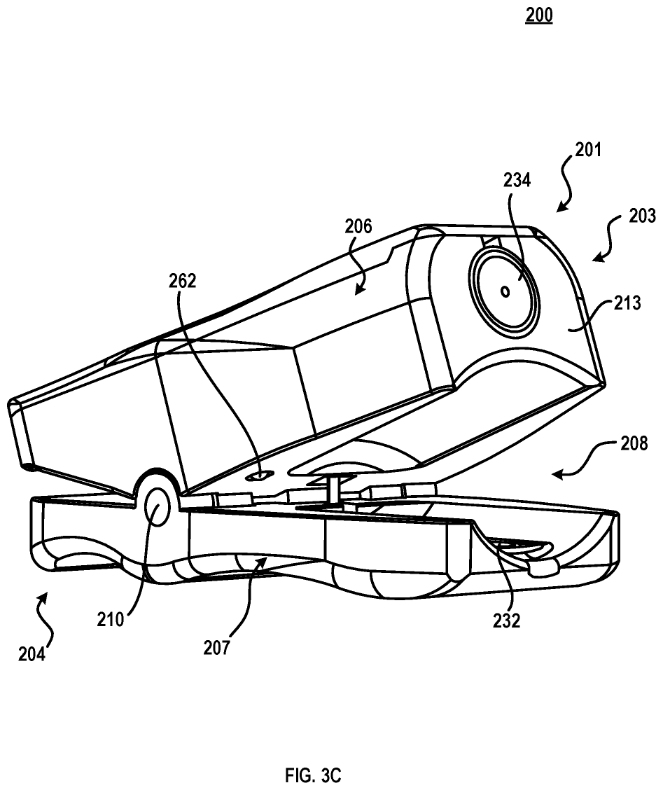

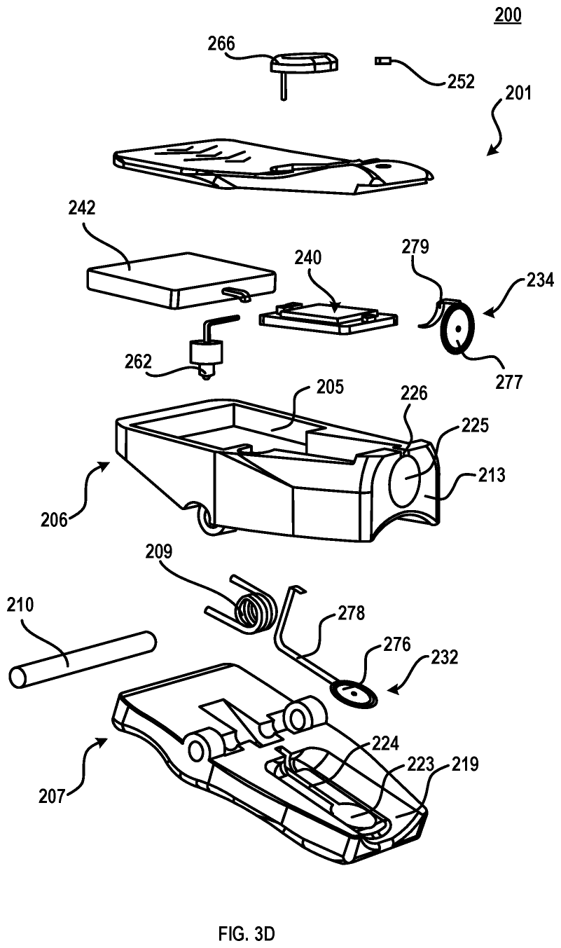

In the electrical embodiment of FIGS. 3A-6G, the device 200 includes a first stimulus element 232 and a second stimulus element 234 that are operable to apply an electrical stimulus to the patient when in contact with the patient. In this case, the first stimulus element 232 and the second stimulus element 234 are each an electrical contact operable to apply a stimulating voltage at the skin of the patient. The device 200 includes a stimulus generator device 230, which can be a waveform generator, and can also include an amplifier 239 that controls an output voltage applied to the first stimulus element 232 and/or the second stimulus element 234 and a monitoring component 249 that monitors voltage values being applied across the first stimulus element 232 and the second stimulus element 234.

In the embodiment of FIGS. 3A-6G, a housing 201 of the device 200 is configured as discussed above with reference to the housing 101 of FIGS. 1A-1F. In the examples of FIGS. 3A-6G, the housing 201 includes a first body portion 206 and a second body portion 207 that collectively form a proximal portion 204 of the device 200 in the same manner discussed above with respect to device 100 and FIGS. 1A-1F, and also collectively form a distal portion 203 of the device 200 in the same manner discussed above with respect to device 100. The distal portion 203 of the device 200 similarly includes a receptacle 208 that captures the digit 10 (FIGS. 1A-1D) of the patient for application of a peripheral stimulus to the digit 10, and also includes a distal-most surface 213 configured to contact a surface 20 (FIGS. 1E and 1F) of the patient for application of a central stimulus to the surface 20 of the patient. As such, the device 200 is configured to transition between a first “closed” configuration shown in FIG. 3A and a second “open” configuration shown in FIG. 3B. As shown in FIGS. 3D, 5A and 5B, the first body portion 206 and the second body portion 207 are connected at a hinge 210 that includes a biasing element 209 that biases the first body portion 206 and the second body portion 207 towards the first “closed” configuration to generate a clamping force that ensures proper contact between the first stimulus element 232 and the digit 10 of the patient when the digit 10 is captured within the receptacle 208 (even when considered “open” due to the presence of the digit 10 within the receptacle 208), and also to ensure that the device 200 remains “closed” when the digit is not captured within the receptacle 208.

With reference to FIGS. 3D-5B, the device 200 includes an electronic assembly 240, which includes a processor 244 and a memory 246 in communication with one or more interfacing components 250. Similar to the one or more interfacing components 150 discussed with reference to device 100 of FIGS. 1A-1F, the one or more interfacing components 250 includes an activation switch 266 for activating the peripheral or central stimulus, a safety switch 264 for preventing accidental discharge, a position switch 262 that controls which of the first stimulus element 232 or the second stimulus element 234 can be activated at a time. The one or more interfacing components 250 can also include one or more indicators 252 that communicate operating data to a practitioner including an on/off state, a stimulating/resting state, an error state, and/or an intensity of the applied stimulus. Further, as shown, the electronic assembly 240 can include a battery 242 that provides power to the electronic assembly 240.

The processor 244 and memory 246 can collectively apply an activating control signal to the stimulus generator device 230 upon receipt of an activation control input from the activation switch 266. The safety switch 264 can electrically decouple an electrical connection between two or more of the processor 244, the stimulus generator device 230, the amplifier 239, and the first stimulus element 232 or the second stimulus element 234 when the safety switch 264 is engaged, thereby causing a voltage at the first stimulus element 232 or the second stimulus element 234 to be zero.

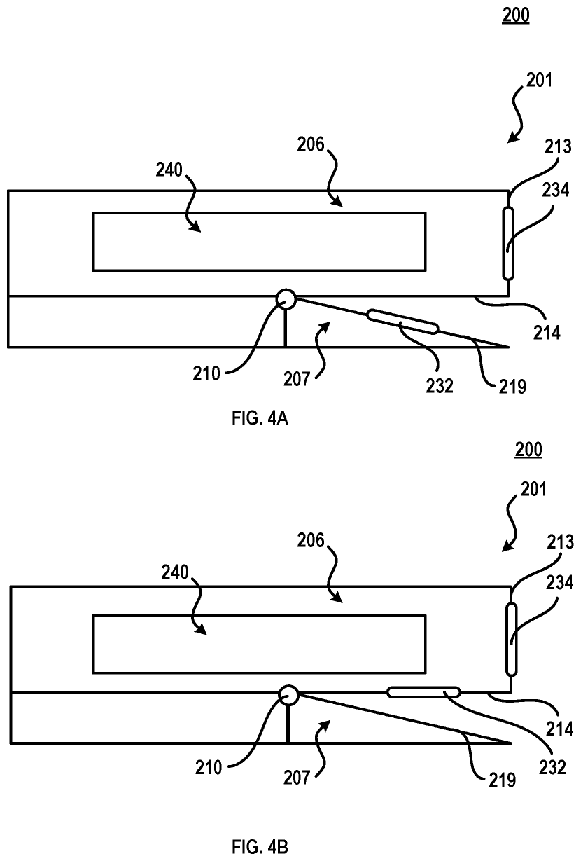

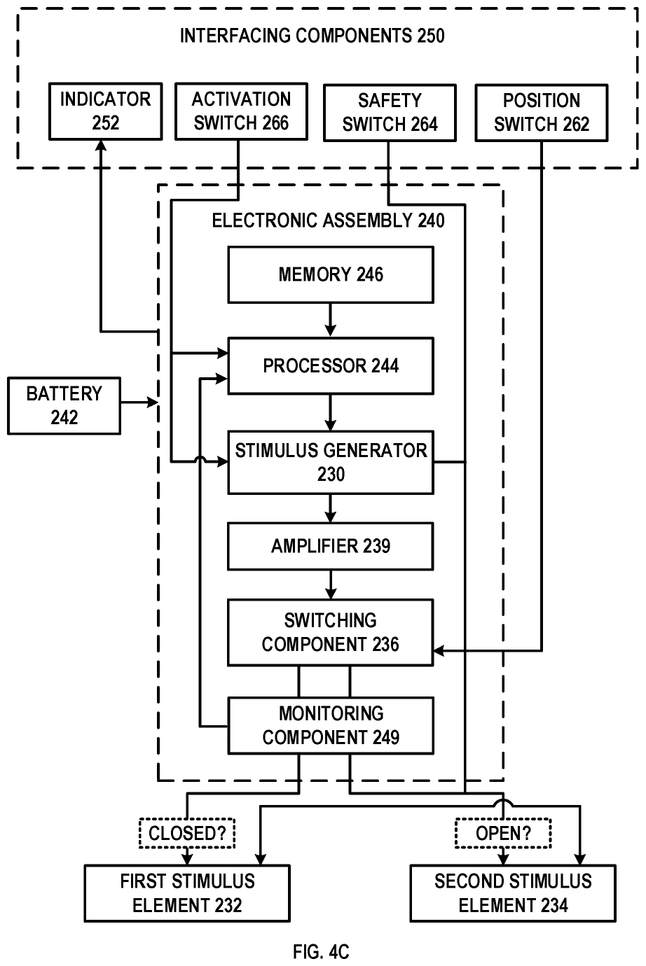

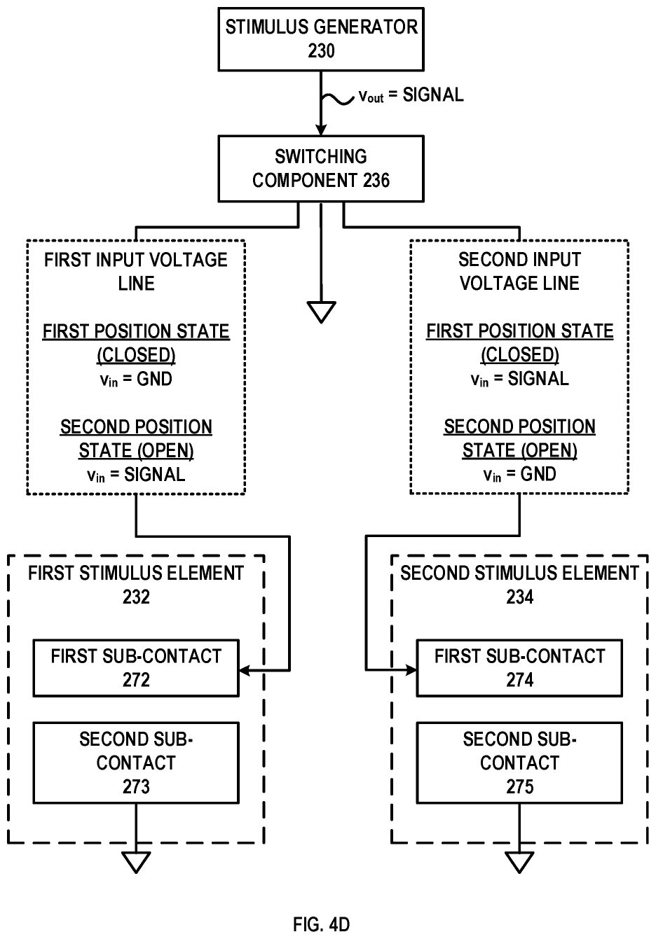

As discussed, the position switch 262 dictates which of the first stimulus element 232 or the second stimulus element 234 is activated at a time. As shown in FIG. 3C, the position switch 262 can be a physical button positioned along a first inward-facing surface 214 of the first body portion 206 that assumes the first positional state (e.g., is “pressed” between the first body portion 206 and the second body portion 207) when the device 200 is in the “closed” configuration (e.g., if a digit is not captured at the receptacle 208) and assumes the second positional state (e.g., is not “pressed” between the first body portion 206 and the second body portion 207) when the device 100 is in the “open” configuration (e.g., if a digit is captured at the receptacle 208). Further, as discussed above and with reference to FIGS. 4C, 4D and 5A, the position switch 262 can communicate with a switching component 236, which can be electrical in nature and can communicate directly with the processor 244 and/or the stimulus generator device 230. The switching component 236 can include digital and/or analog circuitry to electrically decouple the first stimulus element 232 or the second stimulus element 234 from the stimulus generator device 230.

When the position switch 262 is in the first positional state (e.g., by allowing the device 200 to assume the first closed position), the device 200 can allow the second stimulus element 234 positioned along the distal-most surface 213 of the first body portion 206 to activate when the activation switch 266 is activated while preventing activation of the first stimulus element 232 positioned along the receptacle 208. As such, when the first stimulus element 232 is activated, then an input voltage applied to the second stimulus element 234 should be zero voltage. This can be achieved by coupling, by the switching component 236, a second input voltage line of the second stimulus element 234 with a stimulating voltage or “stimulating” line from the stimulus generator device 230 (e.g., the waveform generator) and coupling a first input voltage line of the first stimulus element 232 with a zero voltage or “ground” line when the position switch 262 is in the first positional state.

Similarly, configuring the position switch 262 into the second positional state (e.g., by opening the device 200 to assume the second open position), the device 200 can allow the first stimulus element 232 positioned along the receptacle 208 to activate when the activation switch 266 is activated while preventing activation of the second stimulus element 234 positioned along the distal-most surface 213 of the first body portion 206. As such, when the first stimulus element 232 is activated, then an input voltage applied to the second stimulus element 234 should be zero voltage. This can be achieved by coupling, by the switching component 236, the first input voltage line of the first stimulus element 232 with a stimulating voltage or “stimulating” line and coupling the second input voltage line of the second stimulus element 234 with a zero voltage or “ground” line when the position switch 262 is in the second positional state.

For application of peripheral or central electrical stimuli, the first stimulus element 232 and the second stimulus element 234 can each be an electrode, where the first stimulus element 232 includes at least a first sub-contact 272 and a second sub-contact 273 and where the second stimulus element 234 includes at least a first sub-contact 274 and a second sub-contact 275. Options for electrode designs are presented in FIGS. 6A-6F. As shown in FIGS. 6A and 6B, the first stimulus element 232 and the second stimulus element 234 can each be of a planar concentric configuration, with the first sub-contact 272 of the first stimulus element 232 encircling the second sub-contact 273 of the first stimulus element 232 and with the first sub-contact 274 of the second stimulus element 234 encircling the second sub-contact 275 of the second stimulus element 234. Concentric electrode layouts have been identified as ideal for this application given their ability to stimulate nociceptive A-delta fibers more efficiently than other electrode layouts. Further, in the embodiment of FIGS. 6C and 6D, the first stimulus element 232 and the second stimulus element 234 can each be of a multi-contact planar concentric configuration that include a plurality of planar concentric sub-contacts (each individual planar concentric sub-contact having a respective first and second concentric sub-contact). Similarly, this arrangement includes multiple instances of the first sub-contact 272 of the first stimulus element 232 encircling the second sub-contact 273 of the first stimulus element 232 and the first sub-contact 274 of the second stimulus element 234 encircling the second sub-contact 275 of the second stimulus element 234. Multi-contact planar concentric configuration can elicit even higher selectivity towards A-delta fibers. Benefits include reducing the power consumption, reducing potential for injuring the patient, and reducing potential for damaging nearby medical equipment. One example of the device 200 using the first stimulus element 232 and the second stimulus element 234 of FIGS. 6C and 6D is shown in FIG. 6G. Other options include a planar 2-point configuration shown in FIGS. 6E and 6F, in which the first sub-contact 272 and the second sub-contact 273 of the first stimulus element 232 are positioned adjacent to one another; similarly, the first sub-contact 274 and the second sub-contact 275 of the second stimulus element 234 are positioned adjacent to one another.

Referring back to FIG. 4D, to apply a stimulating voltage at the first stimulus element 232, there must be a voltage differential between the first sub-contact 272 and the second sub-contact 273 of the first stimulus element 232. As such, to apply a stimulating voltage at the first stimulus element 232, a first input voltage line of the first stimulus element 232 can be electrically coupled between the first sub-contact 272 and a stimulating voltage line in electrical communication with the stimulus generator device 230 (e.g., waveform generator), and the second sub-contact 273 can be coupled with a ground voltage line in order to generate a voltage differential between the first sub-contact 272 and the second sub-contact 273 of the first stimulus element 232. Similarly, to apply a stimulating voltage at the second stimulus element 234, there must be a voltage differential between the first sub-contact 274 and the second sub-contact 275 of the second stimulus element 234. As such, to apply a stimulating voltage at the second stimulus element 234, the second input voltage line of the second stimulus element 234 can be electrically coupled between the first sub-contact 274 of the second stimulus element 234 and a stimulating voltage line in electrical communication with the stimulus generator device 230 (e.g., waveform generator), and the second sub-contact 275 can be coupled with a ground voltage line in order to generate a voltage differential between the first sub-contact 274 and the second sub-contact 275 of the second stimulus element 234. The stimulating voltage can be generated at the stimulus generator device 230 and amplified to an appropriate voltage value by the amplifier 239 in accordance with the set of predetermined stimulation parameters. In a primary embodiment, the stimulating voltage generated by the stimulus generator device 230 can be a square wave with an approximate frequency of 100 Hz, however note that other waveform shapes and frequencies can be applied. The stimulus generator device 230 can communicate with the processor 244 to receive the set of predetermined stimulation parameters which can include parameters related to the stimulating voltage including waveform shape, intensity, and frequency. Further, the amplifier 239 can communicate with the processor 244 to receive or otherwise modulate one or more parameters related to the stimulating voltage including a target voltage value of the stimulating voltage.

To return the first stimulus element 232 to a “zero” voltage state, then the processor 244 can cause the stimulus generator device 230 and/or the switching component 236, to apply an input voltage with a value of “zero” or “neutral” at the first input voltage line of the first stimulus element 232 in communication with the first sub-contact 272 of the first stimulus element 232 in order to eliminate the voltage differential between the first sub-contact 272 and the second sub-contact 273 of the first stimulus element 232. Similarly, to return the second stimulus element 234 to a “zero” voltage state, then the processor 244 can cause the stimulus generator device 230 and/or the switching component 236, to apply an input voltage with a value of “zero” or “neutral” at the second input voltage line of the second stimulus element 234 in communication with the first sub-contact 274 of the second stimulus element 234 in order to eliminate the voltage differential between the first sub-contact 274 and the second sub-contact 275 of the second stimulus element 234.

In some embodiments, the first stimulus element 232 is positioned along the second inward-facing surface 219 of the second body portion 207 that forms the second portion of the receptacle 208 as shown in FIG. 4A, however in other embodiments, the first stimulus element 232 can optionally be positioned along the first inward-facing surface 214 of the first body portion 206 that forms the first portion of the receptacle 208 as shown in FIG. 4B. Note that in both configurations, the first stimulus element 232 needs to be in electrical communication with an electronic assembly 240 of the device 200, which is in most cases positioned within an interior cavity 205 of the first body portion 206.

The first stimulus element 232 includes a first element body 276 configured to contact the patient and a first elongated tab portion 278 associated with the stimulus generator device 230. The first elongated tab portion 278 and the first element body 276 can each include a first conductive material for transmission of electrical current from the stimulus generator device 230, through the first elongated tab portion 278, and to the first element body 276 for contact with the digit 10 of the patient. The first sub-contact 272 and the second sub-contact 273 of the first stimulus element 232 are located along the first element body 276 and can be separated from one another by a second insulating material.

As shown in FIG. 3D and corresponding with the example in FIG. 4A, the second inward-facing surface 219 of the second body portion 207 can provide a first recess 223 for placement of the first stimulus element 232 along the second inward-facing surface 219, and can include a first channel 224 for communication of the first elongated tab portion 278 with the interior cavity 205 of the housing 201, particularly within the first body portion 206. As such, the first channel 224 can continue upwards into the first body portion 206 to connect the first elongated tab portion 278 with the stimulus generator device 230. Conversely, in the examples of FIG. 4B, the first inward-facing surface 214 be configured to accept the first stimulus element 232 along the first inward-facing surface 214 of the first body portion 206 and the first elongated tab portion 278 can be positioned within the interior cavity 205 of the first body portion 206 for electrical connection with the stimulus generator device 230.

As shown, the second stimulus element 234 is positioned at the distal-most surface 213 of the first body portion 206 and includes a second element body 277 configured to contact a surface of the patient and a second elongated tab portion 279 associated with the stimulus generator device 230. The second elongated tab portion 279 and the second element body 277 can each include the first conductive material for transmission of electrical current from the stimulus generator device 230, through the second elongated tab portion 279, and to the second element body 277 for contact with the patient. The first sub-contact 274 and the second sub-contact 275 of the second stimulus element 234 are located along the second element body 277 and can be separated from one another by the second insulating material. The distal-most surface 213 provides a second recess 225 for placement of the second stimulus element 234 along the distal-most surface 213, and can include a second channel 226 for communication of the second elongated tab portion 279 with the interior cavity 205, particularly within the first body portion 206.

As discussed above, the stimulus generator device 230 includes a waveform generator; as such, the central stimulus and the peripheral stimulus are each an electrical stimulus generated by the stimulus generator device 230 having a voltage output value and/or a current output value according to the set of predetermined stimulation parameters stored within the memory 246. In a primary embodiment, the voltage output value and/or a current output value can be described as a waveform, which can include a square wave at a frequency of 100 Hz. The first stimulus element 232 can define a first effective surface area (e.g., a geometric area enclosed by the circular shape of the concentric electrode) that applies the peripheral stimulus to a first predefined area corresponding with the first effective surface area along the digit 10 of the patient. Similarly, the second stimulus element 234 can define a second effective surface area (e.g., a geometric area enclosed by the circular shape of the concentric electrode) that applies the central stimulus to a second predefined area along the surface 20 of the patient corresponding with the second effective surface area. As such, a surface area of the applied stimulus is consistent regardless of a size of the patient's body.

In some embodiments, with reference to FIG. 4C, the electronic assembly 240 of the device 200 can include the monitoring component 249 that monitors voltage values being applied across the first stimulus element 232 and the second stimulus element 234 to ensure proper operation of the device 200. In some embodiments, the monitoring component 249 can communicate observed voltage values and other operating data with the processor 244, which can cause the one or more indicators 252 to display an error state if the observed voltage values and other operating data are not within normal operating parameters. For instance, the monitoring component 249 can be configured to measure a first voltage differential between the first sub-contact 272 and the second sub-contact 273 of the first stimulus element 232 and a second voltage differential between the first sub-contact 274 and the second sub-contact 275 of the second stimulus element 234. If the monitoring component 249 detects that the first voltage differential or the second voltage differential are not within an appropriate range, then the processor 244 can cause the one or more indicators 252 to display an error state to the practitioner. Appropriate ranges can be dictated by the set of predetermined stimulation parameters and can also be affected by the positional state of the position switch and/or the activation switch. For instance, if the device 200 is in the second open configuration with the intention of applying a peripheral stimulus to the digit 10 by the first stimulus element 232, but the monitoring component 249 detects a second voltage differential above an appropriate threshold value (when the second voltage differential should be at or near zero), then the processor 244 can cause the one or more indicators 252 to display an error state to the practitioner. In another example, if the device 200 is in the second open configuration with the intention of applying a peripheral stimulus to the digit 10 by the first stimulus element 232, but the monitoring component 249 detects a first voltage differential above or below an appropriate threshold value (above or below the intended voltage value dictated by the set of predetermined stimulation parameters), then the processor 244 can cause the one or more indicators 252 to display an error state to the practitioner. In a further aspect, the monitoring component 249 can report observations to the processor 244, which can be configured to modulate one or more parameters at the stimulus generator device 230 and/or the amplifier 239 to correct any discrepancies.

To apply a peripheral stimulus, a practitioner can open the device 200 to the second open configuration and insert the digit 10 into the receptacle 208 such that the first stimulus element 232 makes substantial contact with the digit 10 and the clamping force generated by the biasing element 209 is applied at the digit 10. By transitioning the device 200 into the second open configuration, the position switch 262 and switching component 236 ensure that only the first stimulus element 232 can activate. If applicable, the practitioner can transition the safety switch 264 into the second safety state (e.g., “safety off”) to enable activation of the first stimulus element 232. The practitioner can then activate the activation switch 266 to activate the stimulus generator device 230 and apply the peripheral stimulus to the digit 10 according to the set of predetermined stimulation parameters stored within the memory 246. The monitoring component 249 can monitor the intensity of the applied stimulus and indicate operating data to the practitioner. It is expected for patients to involuntarily retract their digit 10 upon application of the peripheral stimulus; the device 200 enables the practitioner to maintain their grasp on the digit 10 and ensure effective application of the peripheral stimulus by following, with the device 200, a direction of retraction of the digit 10. Following successful application of the peripheral stimulus, the practitioner can release the activation switch 266 to stop the applied stimulus, and can return the safety switch 264 to the first safety state (e.g., “safety on”).

To apply a central stimulus, a practitioner can close the device 200 to the first closed configuration and contact the surface 20 of the patient with the distal-most surface 213 of the device 200 such that the second stimulus element 234 makes substantial contact with the surface 20. By transitioning the device 100 into the first closed configuration, the position switch 262 and switching component 236 ensure that only the second stimulus element 234 can activate. If applicable, the practitioner can transition the safety switch 264 into the second safety state (e.g., “safety off”) to enable activation of the second stimulus element 234. The practitioner can then activate the activation switch 266 to activate the stimulus generator device 230 and apply the central stimulus to the surface 20 according to the set of predetermined stimulation parameters stored within the memory 246. The monitoring component 249 can monitor the intensity of the applied stimulus and indicate operating data to the practitioner. It is expected for patients to involuntarily retract upon application of the central stimulus; the device 200 enables the practitioner to maintain their contact with the surface 20 and ensure effective application of the central stimulus by following, with the device 200, a direction of retraction of the surface 20. Following successful application of the central stimulus, the practitioner can release the activation switch 266 to stop the applied stimulus, and can return the safety switch 264 to the first safety state (e.g., “safety on”).

The device 200 enables practitioners to rapidly and safely switch between peripheral and central stimulus application in practice. Further, by applying the stimuli according to the set of predetermined stimulation parameters and allowing the practitioner to monitor operating data, the device 200 enables consistent application of the same stimuli each time the device 200 is used, improving the efficacy of assessment methods such as the Glasgow Coma scale.

### Thermal Stimulus

In the embodiment of FIGS. 7A-9C, the device 300 includes a first stimulus element 332 and a second stimulus element 334 are operable to apply a thermal stimulus to the patient when in contact with the patient. In this case, the first stimulus element 332 and the second stimulus element 334 are portals that are each operable to direct a laser to generate a thermal stimulus at the skin of the patient. A stimulus generator device 330 can be a laser generator, and can also include a monitoring component 349 that checks and/or modulates an intensity of the laser applied at the first stimulus element 332 and/or the second stimulus element 334. The stimulus generator device 330 can generate a laser having an output wavelength according to a set of predetermined stimulation parameters, where the laser generates a stimulating temperature along the digit 10 or the surface 20. As such, in the thermal embodiment, the peripheral and central stimuli applied at the patient are resultant of the laser having the output wavelength.

In the embodiment of FIGS. 7A-9C, a housing 301 of the device 300 can be similarly configured as discussed above with reference to the housing 101 of FIGS. 1A-1F, but can include a few key structural differences to accommodate the laser. The device 300 is shown in an assembled state in FIG. 7A. In the examples of FIGS. 7A-9C, the housing 301 includes a first body portion 306 that forms a proximal portion 304 (FIG. 7B) and also part of the distal portion 303 (FIG. 7C) of the device 300; the housing 301 also includes a second body portion 307 that can be substantially shorter than the first body portion 306 and can form part of the distal portion 303 of the device 300. In contrast to the previously-discussed embodiments, the first body portion 306 can be positioned below the second body portion 307, such that a first outward-facing surface 315 forms a bottom surface of the distal portion 303 of the device 300 and a second outward-facing surface 320 forms a top surface of the distal portion 303 of the device 300. The first body portion 306 of the device 300 can include an internal cavity 305 that houses various electronic and optical components of the device 300. The distal portion 303 of the device 300 similarly includes a receptacle 308 that captures the digit 10 (FIGS. 1A-1D) of the patient for application of a peripheral stimulus to the digit 10, and also includes a distal-most surface 313 configured to contact a surface 20 (FIGS. 1E and 1F) of the patient for application of a central stimulus to the surface 20 of the patient. As shown, the receptacle 308 is collectively formed by a first inward-facing surface 314 of the first body portion 306 and a second inward-facing surface 319 of the second body portion 307. In some embodiments, portions of the device 300 can be decoupled from one another and disposed of or cleaned. For instance, as shown in the embodiments of FIGS. 7B and 7C, the distal portion 303 of the device 300 can be decoupled from the proximal portion 304 of the device 300 and the distal portion 303 of the device 300 that contacts the patient can be disposable. Such an arrangement can prevent transmission of infection between patients.

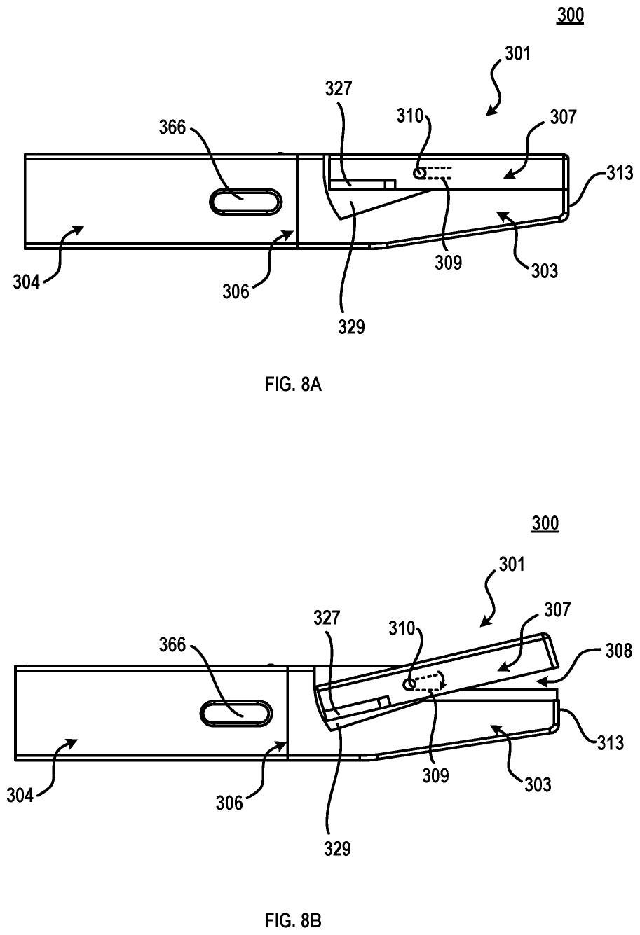

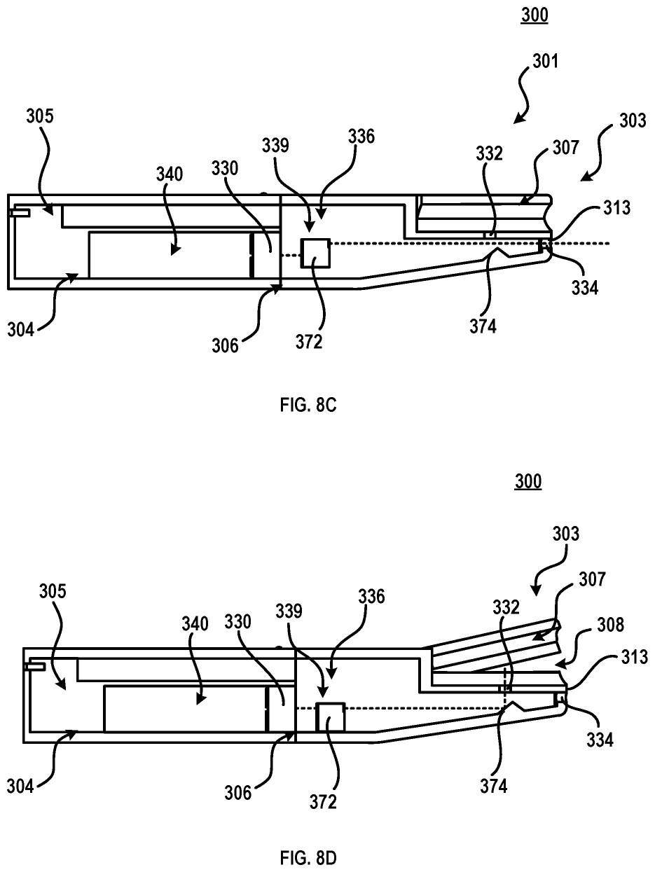

The second body portion 307 can be restricted to the distal portion 303 of the device 300 without forming part of the proximal portion 304 of the device 300. As such, in this arrangement, a hinge 310 that connects the first body portion 306 and the second body portion 307 can be positioned towards the distal portion 303 of the device 300. In this case, the second body portion 307 can include a tab portion 327 that enables a practitioner to transition the device 300 between an “open” configuration (FIGS. 8A and 8C) and a “closed” configuration (FIGS. 8B and 8D). The first body portion 306 can include clearance portions 329 that provide clearance for the second body portion 307 as the second body portion 307 transitions the device 300 between the “open” configuration and the “closed” configuration. In the “open” configuration, the first body portion 306 and the second body portion 307 are forced apart at the distal portion 303 of the device as shown.

In some embodiments, the hinge 310 can include a biasing element 309 that biases the first body portion 306 and the second body portion 307 towards the first “closed” configuration to generate a clamping force that ensures proper contact between the first stimulus element 332 and the digit 10 of the patient when the digit 10 is captured within the receptacle 308 (even when considered “open” due to the presence of the digit 10 within the receptacle 308), and also to ensure that the device 300 remains “closed” when the digit is not captured within the receptacle 308.

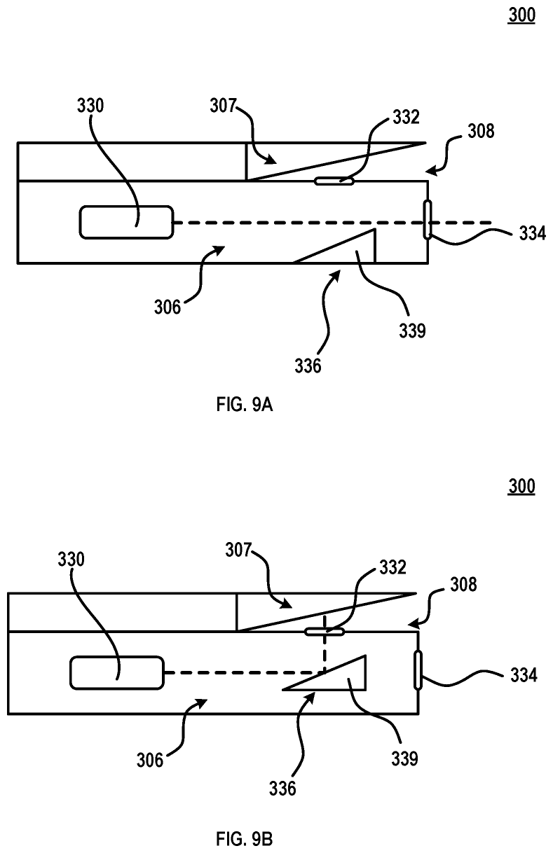

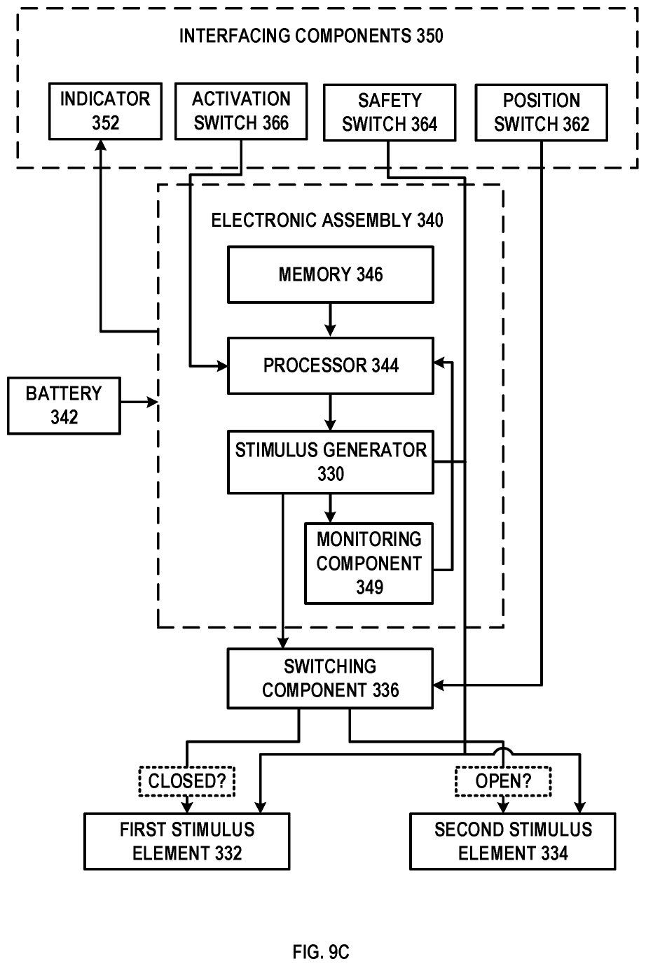

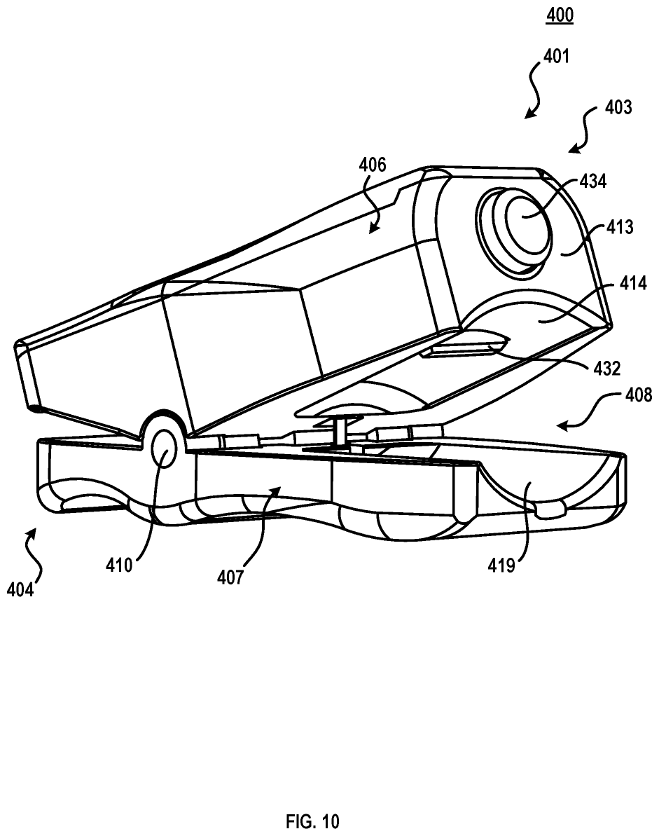

With additional reference to FIGS. 9A-9C, the device 300 includes one or more interfacing components 350 similar to those discussed with reference to the one or more interfacing components 150 discussed with reference to device 100 of FIGS. 2A-2E, including an activation switch 366 for activating the peripheral or central stimulus, a safety switch 364 for preventing accidental discharge, a position switch 362 that controls which of the first stimulus element 332 or the second stimulus element 334 can be activated at a time. The one or more interfacing components 350 can also include one or more indicators 352 that communicate operating data to a practitioner including an on/off state, a stimulating/resting state, an error state, and/or an intensity of the applied stimulus.

The electronic assembly 340 includes a processor 344 in communication with a memory 346 that are collectively operable to apply an activating control signal to the stimulus generator device 330 upon receipt of an activation control input from the activation switch 366. The safety switch 364 can electrically or optically decouple an electrical or optical connection between two or more of the processor 344, the stimulus generator device 330, and the first stimulus element 332 or the second stimulus element 334 when the safety switch 364 is engaged, thereby turning off the laser generated by the stimulus generator device 330 or by otherwise occluding the laser generated by the stimulus generator device 330 such that no light exits the first stimulus element 332 or the second stimulus element 334. In some embodiments, the safety switch 364 can include a simple shutter mechanism that covers one or both of the first stimulus element 332 or the second stimulus element 334 such that the laser does not exit the first stimulus element 332 or the second stimulus element 334. Further, as shown, the electronic assembly 340 can include a battery 342 that provides power to the electronic assembly 340.

The position switch 362 dictates which of the first stimulus element 332 or the second stimulus element 334 is activated at a time. The position switch 362 can be a physical button positioned along the first inward-facing surface 314 of the first body portion 306 that engages (e.g., is “pressed” between the first body portion 306 and the second body portion 307) when the device 300 is in the “closed” configuration (e.g., if a digit is not captured at the receptacle 308) and disengages (e.g., is not “pressed” between the first body portion 306 and the second body portion 307) when the device 300 is in the “open” configuration (e.g., if a digit is captured at the receptacle 308). In some embodiments, the position switch 362 can communicate with a switching component 336 which can include a mirror assembly 339 operable to be configured into a first positional state or a second positional state that diverts a path of the laser out of the first stimulus element 332 or the second stimulus element 334.

As shown with additional reference to FIGS. 8C and 8D, the mirror assembly 339 can be positioned within the first body portion 306 of the device 300 and can include a first mirror sub-assembly 372 and a second mirror sub-assembly 374 that direct the laser between the first stimulus element 332 and the second stimulus element 334. In one embodiment, the first mirror sub-assembly 372 can be moveable between a first vertical position associated with the first positional state and the first closed configuration of the device 300 and a second vertical position associated with the second positional state and the second open configuration of the device 300. As such, when the first mirror sub-assembly 372 is in the first vertical position, the first mirror sub-assembly 372 can direct the laser generated by the stimulus generator device 330 in a horizontal direction along a first latitude. In some embodiments, when in the first mirror configuration, the first mirror sub-assembly 372 allows the laser to travel in the horizontal direction along the first latitude until the laser exits the second stimulus element 334, bypassing the second mirror sub-assembly 374. In contrast, when first mirror sub-assembly 372 is in the second vertical position, the first mirror sub-assembly 372 re-directs the laser to travel in the horizontal direction along the second latitude until the laser reaches the second mirror sub-assembly 374. The second mirror sub-assembly 374 can be configured to re-direct the laser from the horizontal direction along the second latitude to a vertical direction such that the laser exits at the first stimulus element 332.

When the position switch 362 is in the first positional state (e.g., by allowing the device 300 to assume the first closed position), the device 300 can allow the second stimulus element 334 positioned along the distal-most surface 313 of the first body portion 306 to activate when the activation switch 366 is activated while preventing activation of the first stimulus element 332 positioned along the receptacle 308. As such, when the second stimulus element 334 is activated, then there should be zero light emitting from the first stimulus element 332. This can be achieved by directing, by the switching component 336 including the mirror assembly 339, the laser towards the second stimulus element 334 instead of the first stimulus element 332 when the position switch 362 is in the first positional state as discussed above.

Similarly, configuring the position switch 362 into the second positional state (e.g., by opening the device 300 to assume the second open position), the device 300 can allow the first stimulus element 332 positioned along the receptacle 308 to activate when the activation switch 366 is activated while preventing activation of the second stimulus element 334 positioned along the distal-most surface 313 of the first body portion 306. As such, when the first stimulus element 332 is activated, then there should be zero light emitting from the second stimulus element 334. This can be achieved by directing, by the switching component 336 including the mirror assembly 339, the laser towards the first stimulus element 332 instead of the second stimulus element 334 when the position switch 362 is in the second positional state as discussed above.

As discussed above, the stimulus generator device 330 can generate the laser having an output wavelength according to the set of predetermined stimulation parameters stored within the memory 346, where the laser generates a stimulating temperature along the digit 10 or the surface 20. As such, in the thermal embodiment, the peripheral and central stimuli applied at the patient are resultant of the laser having the output wavelength. The first stimulus element 332 and the second stimulus element 334 are laser portals that apply the stimulus to a predefined area along a surface of the digit corresponding with a diameter of the laser. As such, a surface area of the applied stimulus is consistent regardless of a size of the patient's body.

In some embodiments, the electronic assembly 340 of the device 300 can include the monitoring component 349 that monitors an intensity of the laser being applied at the first stimulus element 332 and/or the second stimulus element 334 to ensure proper operation of the device 300. In some embodiments, the monitoring component 349 can communicate observed light intensity values and other operating data with the processor 344, which can cause the one or more indicators 352 to display an error state if the light intensity values and other operating data are not within normal operating parameters. If the monitoring component 349 detects that the observed light intensity values are not within an appropriate range, then the processor 344 can cause the one or more indicators 352 to display an error state to the practitioner. Appropriate ranges can be dictated by the set of predetermined stimulation parameters and can also be affected by the positional state of the position switch and/or the activation switch. For instance, if the device 300 is in the second open configuration with the intention of applying a peripheral stimulus to the digit 10 by the first stimulus element 332, but the monitoring component 349 detects an observed light intensity value that is above or below an appropriate threshold value dictated by the set of predetermined stimulation parameters, then the processor 344 can cause the one or more indicators 352 to display an error state to the practitioner and/or modulate one or more parameters applied at the stimulus generator device 330.

To apply a peripheral stimulus, a practitioner can open the device 300 to the second open configuration and insert the digit 10 into the receptacle 308 such that the first stimulus element 332 makes substantial contact with the digit 10 and the clamping force generated by the biasing element 309 is applied at the digit 10. By transitioning the device 300 into the second open configuration, the position switch 362 and switching component 336 ensure that only the first stimulus element 332 can activate. If applicable, the practitioner can transition the safety switch 364 into the second safety state (e.g., “safety off”) to enable activation of the first stimulus element 332. The practitioner can then activate the activation switch 366 to activate the stimulus generator device 330 and apply the peripheral stimulus to the digit 10 according to the set of predetermined stimulation parameters stored within the memory 346. The monitoring component 349 can monitor the intensity of the applied stimulus and indicate operating data to the practitioner. It is expected for patients to involuntarily retract their digit 10 upon application of the peripheral stimulus; the device 300 enables the practitioner to maintain their grasp on the digit 10 and ensure effective application of the peripheral stimulus by following, with the device 300, a direction of retraction of the digit 10. Following successful application of the peripheral stimulus, the practitioner can release the activation switch 366 to stop the applied stimulus, and can return the safety switch 364 to the first safety state (e.g., “safety on”).

To apply a central stimulus, a practitioner can close the device 300 to the first closed configuration and contact the surface 20 of the patient with the distal-most surface 313 of the device 300 such that the second stimulus element 334 makes substantial contact with the surface 20. By transitioning the device 300 into the first closed configuration, the position switch 362 and switching component 336 ensure that only the second stimulus element 334 can activate. If applicable, the practitioner can transition the safety switch 364 into the second safety state (e.g., “safety off”) to enable activation of the second stimulus element 334. The practitioner can then activate the activation switch 366 to activate the stimulus generator device 330 and apply the central stimulus to the surface 20 according to the set of predetermined stimulation parameters stored within the memory 346. The monitoring component 349 can monitor the intensity of the applied stimulus and indicate operating data to the practitioner. It is expected for patients to involuntarily retract upon application of the central stimulus; the device 300 enables the practitioner to maintain their contact with the surface 20 and ensure effective application of the central stimulus by following, with the device 300, a direction of retraction of the surface 20. Following successful application of the central stimulus, the practitioner can release the activation switch 366 to stop the applied stimulus, and can return the safety switch 364 to the first safety state (e.g., “safety on”).

The device 300 and enables practitioners to switch between peripheral and central stimulus application rapidly and safely. Further, by applying the stimuli according to the set of predetermined stimulation parameters and allowing the practitioner to monitor operating data, the device 300 enables consistent application of the same stimuli each time the device 300 is used, improving the efficacy of assessment methods such as the Glasgow Coma scale.

### Mechanical Stimulus

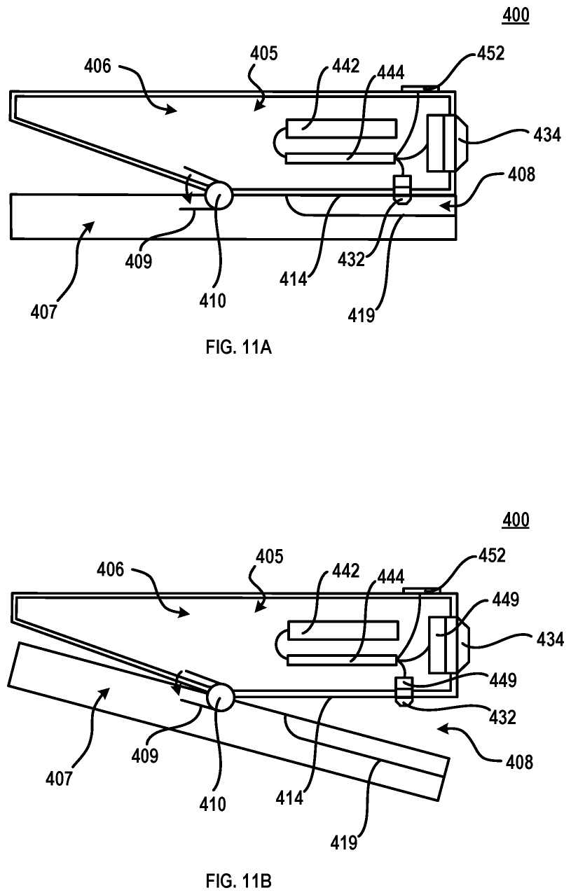

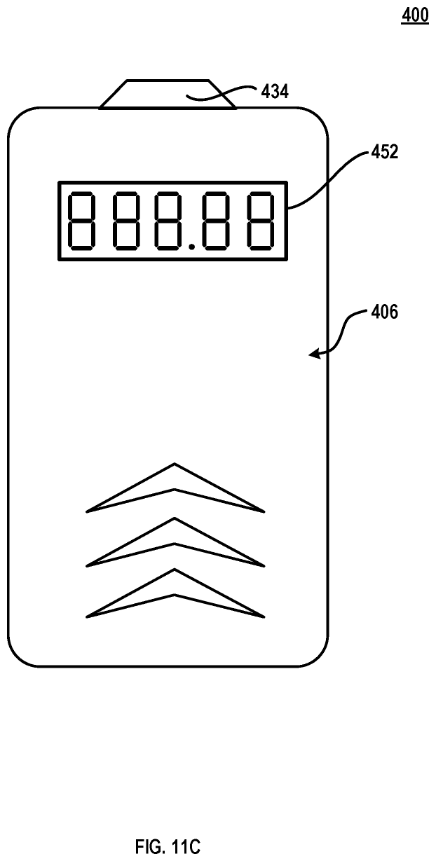

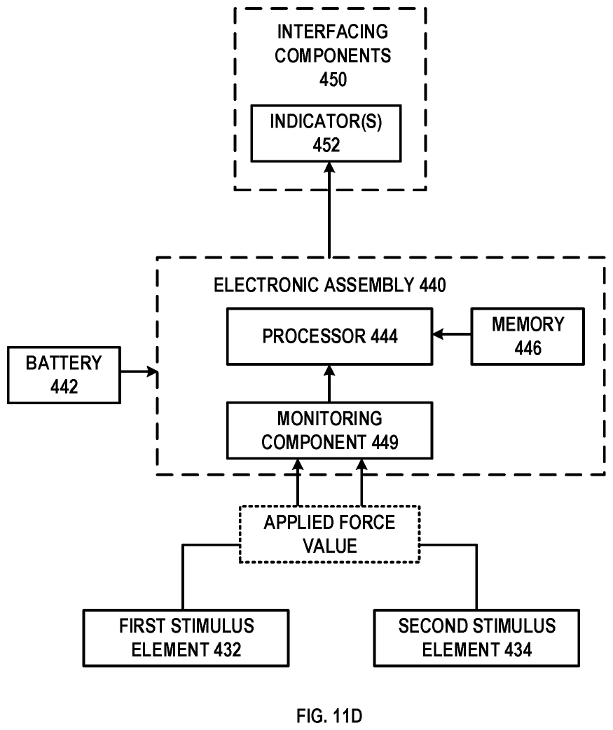

In the embodiment of FIGS. 10-12C, the device 400 includes a first stimulus element 432 and a second stimulus element 434 which are operable to apply a mechanical stimulus to the patient when in contact with the patient. In this case, the first stimulus element 432 and the second stimulus element 434 are each a force applicator operable to apply a stimulating mechanical force at the skin of the patient. For the examples in FIGS. 11A-11D, a first body portion 406 of the device 400 can include an internal cavity 405 that houses an electronic assembly 440 of the device 400 including a battery 442 and a processor 444.

To apply a peripheral stimulus or a central stimulus to the patient using the device 400, a practitioner can press the first stimulus element 432 or the second stimulus element 434 against the digit 10 or the surface 20 of the patient to generate the stimulus in the form of a mechanical force. The device 400 can include a monitoring component 449 to monitor the mechanical force applied to the first stimulus element 432 and/or the second stimulus element 434. In particular, the first stimulus element 432 and the second stimulus element 434 can apply a physical pressure to the patient via force applicators, which can be in the form of movable protrusions that focus a mechanical force at the digit 10 or the surface 20 of the patient. The force applied at the first stimulus element 432 and the second stimulus element 434 can be an instantaneous force or can be a force applied over a few seconds. In the embodiment shown, the first stimulus element 432 can be configured to apply the peripheral stimulus in the form of a mechanical force along a nail bed at the digit 10 of the patient, and can be configured in a bar shape as shown. The first stimulus element 432 can define a first surface area that applies the peripheral stimulus along a first predetermined area along the digit 10. The second stimulus element 434 can be configured to apply the central stimulus in the form of a mechanical force along a supraorbital notch at the surface 20 of the patient, and can be configured in a circular shape as shown. Similarly, the second stimulus element 434 can define a second surface area that applies the central stimulus along a second predetermined area along the surface 20 of the patient.

In the embodiment of FIGS. 10-12C, a housing 401 of the device 400 is configured similarly as discussed above with reference to the housing 101 of FIGS. 1A-1F. In the examples of FIGS. 10-12C, the first body portion 406 and the second body portion 407 collectively form a proximal portion 404 of the device 400 in the same manner discussed above with respect to device 100, and also collectively form a distal portion 403 of the device 400 in the same manner discussed above with respect to device 100. The distal portion 403 of the device 400 similarly includes a receptacle 408 collectively formed by a first inward-facing surface 414 of the first body portion 406 and a second inward-facing surface 419 of the second body portion 407 that captures the digit 10 (FIGS. 1A-1D) of the patient for application of a peripheral stimulus to the digit 10. The distal portion 403 of the device 400 also includes a distal-most surface 413 configured to contact the surface 20 (FIGS. 1E and 1F) of the patient for application of a central stimulus to the surface 20 of the patient. However, note that the device 400 of FIGS. 10-12C requires placement of the first stimulus element 432 along a first inward-facing surface 414 of the first body portion 406 (as the first stimulus element 432 must interface with a nail bed along a top of the digit 10). As shown, the second stimulus element 434 is positioned at the distal-most surface 413 of the first body portion 406.

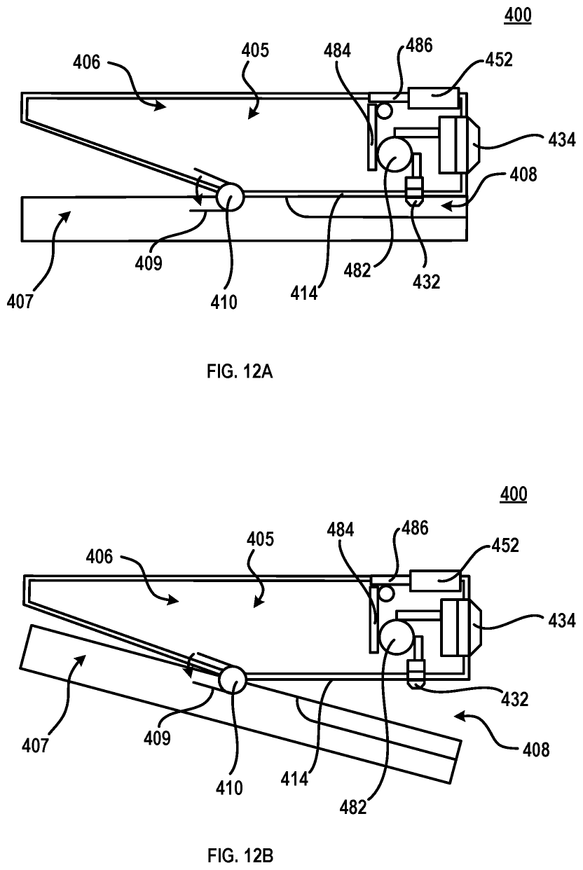

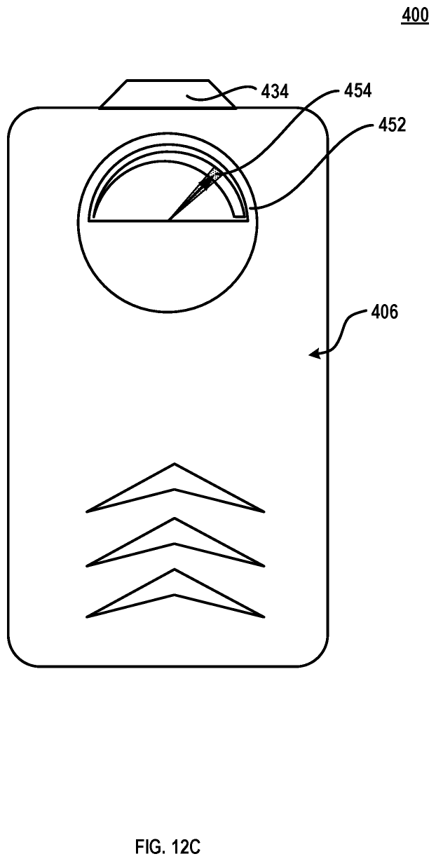

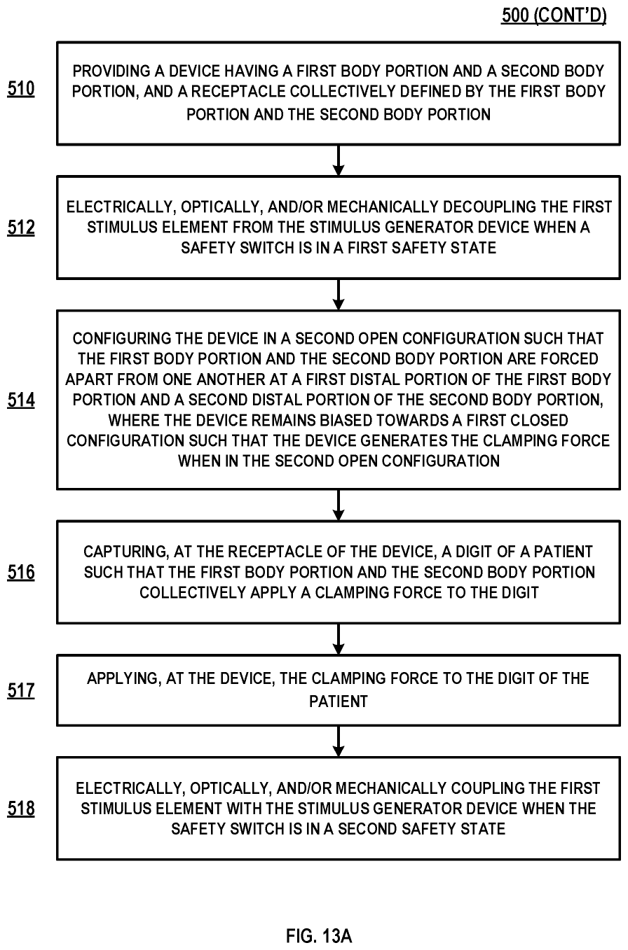

Similar to device 100 of FIGS. 1A-1F, the device 400 is configured to transition between a first “closed” configuration shown in FIGS. 11A and 12A and a second “open” configuration shown in FIGS. 11B and 12B. As shown, the first body portion 406 and the second body portion 407 are connected at a hinge 410 that includes a biasing element 409 that biases the first body portion 406 and the second body portion 407 towards the first “closed” configuration to generate a clamping force that ensures proper contact between the first stimulus element 432 and the digit 10 of the patient when the digit 10 is captured within the receptacle 408 (even when considered “open” due to the presence of the digit 10 within the receptacle 408), and also to ensure that the device 100 remains “closed” when the digit is not captured within the receptacle 408.

The device 400 includes one or more interfacing components 450 including one or more indicators 452 that communicate an intensity of the applied stimulus to the practitioner. In some embodiments, the device 400 can include the monitoring component 449 that monitors force values being applied at the first stimulus element 432 and the second stimulus element 434 to ensure proper operation of the device 400. In some embodiments, the monitoring component 449 can measure and communicate observed force values and other operating data with the processor 444, which can then be displayed at the one or more indicators 452 for the practitioner's knowledge. In the embodiment of FIGS. 11A-11D, the monitoring component 449 can include one or more electronic force sensors. The processor can cause the one or more indicators 452 to display an error state if the monitoring component 449 is unable to measure the applied force. Further, the processor can cause the one or more indicators 452 to inform the practitioner when an appropriate force value is detected at the first stimulus element 432 or the second stimulus element 434. For instance, the monitoring component 449 can be configured to measure a first force value applied at first stimulus element 432 and a second force value applied at the second stimulus element 434. If the monitoring component 449 detects that the first force value or the second force value are within an appropriate range, then the processor 444 can cause the one or more indicators 452 to display an affirmative message to the practitioner, which can be in the form of a color change (e.g., green for force values within the appropriate range, red for force values that are not within the appropriate range), “blinking” the one or more indicators 452. Appropriate ranges can be dictated by the set of predetermined stimulation parameters and can also be affected by the positional state of the position switch and/or the activation switch. In the example shown in FIG. 11C, the one or more indicators 452 can be a 7-segment display.

In some embodiments shown in FIGS. 12A-12C, the monitoring component 449 and indicators 452 can be mechanical in nature to provide accurate readings of applied force without the need for more complicated electronic force sensors. Such an arrangement can include an applicator pinion 482 in association with the first stimulus element 432 and/or the second stimulus element 434 that rotates in response to forces being applied at the first stimulus element 432 and/or the second stimulus element 434. The monitoring component 449 can further include a secondary gear rack 484 positioned between the applicator pinion 482 and a gauge gear rack 486 to transfer rotational force generated at the applicator pinion 482 to a linear force interpretable by the gauge gear rack 486. The gauge gear rack 486 is associated with the one or more indicators 452, which can include an analog force indicator shown in FIG. 12C. As shown, the one or more indicators 452 can include a marker 454 that indicates a “target” range associated with the set of predetermined stimulation parameters.

To apply a peripheral stimulus, a practitioner can open the device 400 to the second open configuration and insert the digit 10 into the receptacle 408 such that the first stimulus element 432 makes substantial contact with the digit 10 and the clamping force generated by the biasing element 109 is applied at the digit 10. The practitioner can then manually “squeeze” the first body portion 406 and the second body portion 407 together at the distal portion 403 of the device 400 to apply the peripheral stimulus to the digit 10 until the indicator 452 indicates that an intensity of the applied stimulus is within a target range dictated by the set of predetermined stimulation parameters. The monitoring component 449 can monitor the intensity of the applied stimulus and indicate operating data to the practitioner. In embodiments where the monitoring component 449 is electronic, the monitoring component 449 can compare the intensity of the applied stimulus with a set of predetermined stimulation parameters stored within a memory 446 and inform the practitioner if they need to apply more or less force. In embodiments where the monitoring component 449 is analog in nature, the indicator 452 can display the target range of applied force values by the marker 454. It is expected for patients to involuntarily retract their digit 10 upon application of the peripheral stimulus; the device 400 enables the practitioner to maintain their grasp on the digit 10 and ensure effective application of the peripheral stimulus by following, with the device 400, a direction of retraction of the digit 10.

To apply a central stimulus, a practitioner can close the device 400 to the first closed configuration and contact the surface 20 of the patient with the distal-most surface 413 of the device 400 such that the second stimulus element 434 makes substantial contact with the surface 20. The practitioner can then manually “press” the first body portion 406 against the surface 20 to apply the peripheral stimulus to the surface 20, increasing force until the indicator 452 indicates that an intensity of the applied stimulus is within a target range dictated by the set of predetermined stimulation parameters. The monitoring component 449 can monitor the intensity of the applied stimulus and indicate operating data to the practitioner. In embodiments where the monitoring component 449 is electronic, the monitoring component 449 can compare the intensity of the applied stimulus with a set of predetermined stimulation parameters stored within a memory 446 and inform the practitioner if they need to apply more or less force. In embodiments where the monitoring component 449 is analog in nature, the indicator 452 can display the target range of applied force values by the marker 454. It is expected for patients to involuntarily retract upon application of the central stimulus; the device 400 enables the practitioner to maintain their contact with the surface 20 and ensure effective application of the peripheral stimulus by following, with the device 400, a direction of retraction of the surface 20.

The handheld profile of the device 400 enables practitioners to rapidly and safely switch between peripheral and central stimulus application in practice. Further, by applying the stimuli according to the set of predetermined stimulation parameters and allowing the practitioner to monitor operating data, the device 400 enables consistent application of the same stimuli each time the device 400 is used, improving the efficacy of assessment methods such as the Glasgow Coma scale.

### Methods

With reference to FIGS. 13A and 13B, a first method 500 is illustrated for applying a peripheral stimulus to a digit of a patient using the device 100, 200, 300, and 400. Similarly, FIGS. 14A and 14B show a second method 600 for applying a central stimulus to a surface of a patient using the device 100, 200, 300, and 400. Note that in practice, the device 100, 200, 300, and 400 enables a practitioner to alternate between application of peripheral and central stimuli; as such, steps of methods 500 and 600 can be applied by the practitioner in a suitable order.

Referring first to FIGS. 13A and 13B, to apply a peripheral stimulus, step 510 of method 500 includes providing a device (e.g., device 100, 200, 300, and 400) having a first body portion and a second body portion, and a receptacle collectively defined by the first body portion and the second body portion. Step 512 includes electrically, optically, and/or mechanically decoupling a first stimulus element from a stimulus generator device when a safety switch is in a first safety state (e.g., “safety on”) when the device is not in use. When it is time to apply the stimulus, step 514 includes configuring the device in a second open configuration such that the first body portion and the second body portion are forced apart from one another at a first distal portion of the first body portion and a second distal portion of the second body portion, where the device remains biased towards a first closed configuration such that the device generates a clamping force by a biasing element when in the second open configuration. Step 516 includes capturing, at a receptacle of the device, a digit of a patient such that the first body portion and the second body portion collectively apply the clamping force to the digit. For application of peripheral stimuli, step 517 includes applying, at the device, the clamping force generated by a biasing element to the digit of the patient. Step 518 includes electrically, optically, and/or mechanically coupling the first stimulus element with the stimulus generator device when the safety switch is in a second safety state (e.g., “safety off”).

Step 520 includes activating the stimulus generator device when an activation switch is in a first activation state. Step 522 includes applying, at the first stimulus element in communication with the stimulus generator device and positioned along the receptacle, a peripheral stimulus to the digit based on a set of predetermined stimulation parameters. Application of the peripheral stimulus may elicit an involuntary reaction from the patient in which the patient retracts, or pulls back, the digit in response to the peripheral stimulus. Step 524 includes following, by the device, a direction of retraction of the digit of the patient following initial application of the peripheral stimulus at the digit of the patient. The “clamping” mechanism and portable profile provided by the device enables the practitioner to follow the direction of retraction to ensure continued application of the peripheral stimulus. Step 526 includes deactivating the stimulus generator device when the activation switch is in a second activation state following application of the peripheral stimulus to the patient; this step can also include electrically, optically, and/or mechanically decoupling a first stimulus element from a stimulus generator device when a safety switch is in a first safety state (e.g., “safety on”).

Referring now to FIGS. 14A and 14B, to apply a central stimulus, step 610 of method 600 includes providing a device (e.g., device 100, 200, 300, and 400) having a first body portion and a second body portion, and a receptacle collectively defined by the first body portion and the second body portion. Step 612 includes electrically, optically, and/or mechanically decoupling the second stimulus element from the stimulus generator device when a safety switch is in a first safety state when the device is not in use (e.g., “safety on”). When it is time to apply the stimulus, step 614 includes configuring the device in a first closed configuration such that a first distal portion of the first body portion is in substantial contact with a second distal portion of the second body portion. Step 616 includes contacting, at the distal-most surface of the device, a surface of a patient such that the second stimulus element contacts the surface of the patient. Step 618 includes electrically, optically, and/or mechanically coupling the second stimulus element with the stimulus generator device when the safety switch is in a second safety state (e.g. “safety off”).

Step 620 includes activating the stimulus generator device when an activation switch is in a first activation state. Step 622 includes applying, at a second stimulus element in communication with the stimulus generator device and positioned along the first body portion, a central stimulus to a surface of the patient based on the set of predetermined stimulation parameters. Application of the peripheral stimulus may elicit an involuntary reaction from the patient in which the patient retracts, or pulls back, the digit in response to the peripheral stimulus. Step 624 includes following, by the device, a direction of retraction of the surface of the patient following initial application of the central stimulus at the surface of the patient. The portable profile provided by the device enables the practitioner to follow the direction of retraction to ensure continued application of the peripheral stimulus. Step 626 includes deactivating the stimulus generator device when the activation switch is in a second activation state; this step can also include electrically, optically, and/or mechanically decoupling a first stimulus element from a stimulus generator device when a safety switch is in a first safety state (e.g., “safety on”).

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A device, comprising:
a housing defining a first body portion and a second body portion, the first body portion and the second body portion collectively defining a receptacle;
wherein the receptacle is configured to capture a digit of a patient therein; and
wherein the first body portion and the second body portion collectively apply a clamping force to the digit;
a stimulus generator device positioned within the housing and being operable to generate a stimulus based on a set of predetermined stimulation parameters; and
a first stimulus element in communication with the stimulus generator device and positioned along the receptacle, the first stimulus element being operable to apply the stimulus to the digit.

2. The device of claim 1, wherein the first body portion and the second body portion are operable to collectively assume a first closed configuration and a second open configuration.

3. The device of claim 2, wherein the stimulus generator device is operable to apply the stimulus to a surface of the digit through the first stimulus element when the first body portion and the second body portion are in the second open configuration.

4. The device of claim 2, wherein the housing includes a biasing element that biases the first body portion and the second body portion towards the first closed configuration and generates the clamping force applied to the digit when the first body portion and the second body portion are in the second open configuration.

5. The device of claim 1, wherein the stimulus generator device includes a waveform generator and wherein the stimulus is an electrical stimulus generated by the waveform generator having a voltage output value and/or a current output value according to the set of predetermined stimulation parameters.

6. The device of claim 5, wherein the first stimulus element is an electrode having an effective surface area that applies the stimulus to a predefined area along a surface of the digit corresponding with the effective surface area.

7. The device of claim 1, wherein the stimulus generator device includes a laser generator that generates a laser having an output wavelength according to the set of predetermined stimulation parameters, wherein the stimulus is a thermal stimulus generating a stimulating temperature along a surface of the digit and wherein the stimulus is resultant of the laser having the output wavelength.

8. The device of claim 7, wherein the first stimulus element is a laser portal that applies the stimulus to a predefined area along the digit corresponding with a diameter of the laser.

9. The device of claim 1, wherein the housing is portable and wherein the first body portion and/or the second body portion include a gripping portion.

10. The device of claim 1, wherein the first body portion includes a distal-most surface, the distal-most surface including a second stimulus element in communication with the stimulus generator device.

11. The device of claim 10, wherein the stimulus generator device is operable to apply the stimulus to a surface of the patient through the second stimulus element when the first body portion and the second body portion are in a first closed configuration.

12. The device of claim 11, further comprising:
a position switch positioned along the first body portion and configured to assume a first position state when the first body portion and the second body portion are in the first closed configuration and configured to assume a second position state when the first body portion and the second body portion are in a second open configuration;
wherein the position switch causes the second stimulus element to apply the stimulus when the position switch is in the first position state; and
wherein the position switch causes the first stimulus element to apply the stimulus when the position switch is in the second position state.

13. The device of claim 1, further comprising an activation switch operable for assuming a first activation state and a second activation state, wherein the activation switch is operable for activating the stimulus generator device when in the first activation state and wherein the activation switch is operable for deactivating the stimulus generator device when in the second activation state.

14. A device, comprising:
a housing defining a first body portion and a second body portion, the first body portion and the second body portion collectively defining a receptacle;
wherein the first body portion and the second body portion are operable to assume a first closed configuration and a second open configuration;
wherein the receptacle is configured to capture a digit of a patient therein when in the second open configuration; and
wherein the first body portion and the second body portion collectively apply a clamping force to the digit when in the second open configuration;
a stimulus generator device housed within the housing and configured to generate a central stimulus and a peripheral stimulus based on a set of predetermined stimulation parameters;
a second stimulus element in communication with the stimulus generator device and positioned along the first body portion, the second stimulus element being operable to apply the central stimulus to a surface of the patient when in the first closed configuration; and
a first stimulus element in communication with the stimulus generator device and positioned along the receptacle, the first stimulus element being operable to apply the peripheral stimulus to the digit when in the second open configuration.

15. The device of claim 14, wherein the housing includes a biasing element that biases the first body portion and the second body portion towards the first closed configuration and generates the clamping force applied to the digit when the first body portion and the second body portion are in the second open configuration.

16. The device of claim 14, wherein the stimulus generator device includes a waveform generator and wherein the central stimulus and the peripheral stimulus are each an electrical stimulus generated by the waveform generator having a voltage output value and/or a current output value according to the set of predetermined stimulation parameters.

17. The device of claim 16, wherein the first stimulus element is an electrode having a first effective surface area that applies the peripheral stimulus to a first predefined area corresponding with the first effective surface area along the digit of the patient and wherein the second stimulus element is an electrode having a second effective surface area that applies the central stimulus to a second predefined area along a surface of the patient corresponding with the second effective surface area.

18. The device of claim 14, wherein the stimulus generator device includes a laser generator that generates a laser having an output wavelength according to the set of predetermined stimulation parameters, wherein the peripheral stimulus and the central stimulus are each a thermal stimulus having a stimulating temperature applied at a surface of the patient and wherein the peripheral stimulus and the central stimulus are resultant of the laser having the output wavelength.

19. The device of claim 18, wherein the first stimulus element is a laser portal that applies the central stimulus to a first predefined area corresponding with a first diameter of the laser along a surface of the digit of the patient and the second stimulus element is a laser portal that applies the peripheral stimulus to a second predefined area corresponding with a second diameter of the laser along the surface of the patient.

20. The device of claim 14, wherein the housing is portable and wherein the first body portion and/or the second body portion include a gripping portion.

21. The device of claim 14, further comprising:
a position switch positioned along a surface of the first body portion and configured to assume a first position state when the first body portion and the second body portion are in the first closed configuration and further configured to assume a second position state when the first body portion and the second body portion are in the second open configuration;
wherein the position switch causes the second stimulus element to apply the central stimulus when the position switch is in the first position state; and
wherein the position switch causes the first stimulus element to apply the peripheral stimulus when the position switch is in the second position state.

22. The device of claim 21, wherein the central stimulus and the peripheral stimulus are each an electrical stimulus and wherein the position switch causes the first stimulus element to have a minimal voltage between a first sub-contact and a second sub-contact of the first stimulus element when the position switch is in the first position state and wherein the position switch causes the second stimulus element to have a minimal voltage between a first sub-contact and a second sub-contact of the second stimulus element when the position switch is in the second position state.

23. The device of claim 21, wherein the central stimulus and the peripheral stimulus are each a thermal stimulus resultant of a laser generated at the stimulus generator device and wherein the position switch causes the first stimulus element to emit minimal light when the position switch is in the first position state and wherein the position switch causes the second stimulus element to emit minimal light when the position switch is in the second position state.

24. The device of claim 23, further comprising a mirror assembly positioned within the first body portion that diverts a path of the laser towards the second stimulus element when the position switch is in the first position state and diverts a path of the laser towards the first stimulus element when the position switch is in the second position state.

25. The device of claim 14, further comprising a safety switch operable for assuming a first safety state and a second safety state, wherein the safety switch is operable for electrically, optically, and/or mechanically decoupling the first stimulus element and/or the second stimulus element from the stimulus generator device when the safety switch is in the first safety state and wherein the safety switch is operable for electrically, optically, and/or mechanically coupling the first stimulus element and/or the second stimulus element with the stimulus generator device when the safety switch is in the second safety state.

26. The device of claim 14, further comprising an activation switch operable for assuming a first activation state and a second activation state, wherein the activation switch is operable for activating the stimulus generator device when in the first activation state and wherein the activation switch is operable for deactivating the stimulus generator device when in the second activation state.

27. The device of claim 26, wherein the activation switch includes a first activation sub-switch and a second activation sub-switch, wherein the activation switch requires simultaneous activation of the first activation sub-switch and the second activation sub-switch to assume the first activation state.

28. A method, comprising:
providing a device having a first body portion and a second body portion, and a receptacle collectively defined by the first body portion and the second body portion;
capturing, at the receptacle of the device, a digit of a patient such that the first body portion and the second body portion collectively apply a clamping force to the digit; and
applying, at a first stimulus element in communication with a stimulus generator device and positioned along the receptacle, a peripheral stimulus to the digit based on a set of predetermined stimulation parameters.

29. The method of claim 28, further comprising:
applying, at a second stimulus element in communication with the stimulus generator device and positioned along the first body portion, a central stimulus to a surface of the patient based on the set of predetermined stimulation parameters.

30. The method of claim 29, further comprising:
electrically, optically, and/or mechanically decoupling the first stimulus element and/or the second stimulus element from the stimulus generator device when a safety switch is in a first safety state; and
electrically, optically, and/or mechanically coupling the first stimulus element and/or the second stimulus element with the stimulus generator device when the safety switch is in a second safety state.

31. The method of claim 28, further comprising:
configuring the device in a first closed configuration such that a first distal portion of the first body portion is in substantial contact with a second distal portion of the second body portion.

32. The method of claim 28, further comprising:
configuring the device in a second open configuration such that the first body portion and the second body portion are forced apart from one another at a first distal portion of the first body portion and a second distal portion of the second body portion, wherein the device remains biased towards a first closed configuration such that the device generates the clamping force when in the second open configuration.

33. The method of claim 28, further comprising:
applying, at the device, the clamping force to the digit of the patient; and
following, by the device, a direction of retraction of the digit of the patient following initial application of the peripheral stimulus at the digit of the patient.

34. The method of claim 28, further comprising:
activating the stimulus generator device when an activation switch is in a first activation state; and
deactivating the stimulus generator device when the activation switch is in a second activation state.

35. A device, comprising:
a housing defining a first body portion and a second body portion, the first body portion and the second body portion collectively defining a receptacle;
wherein the first body portion and the second body portion are operable to assume a first closed configuration and a second open configuration;
wherein the receptacle is configured to capture a digit of a patient therein when in the second open configuration; and
wherein the first body portion and the second body portion collectively apply a clamping force to the digit when in the second open configuration;
a first stimulus element in communication and positioned along the receptacle, the first stimulus element being operable to apply a peripheral stimulus to the digit when in the second open configuration; and
a second stimulus element positioned along the first body portion, the second stimulus element being operable to apply a central stimulus to a surface of the patient when in the first closed configuration.

36. The device of claim 35, wherein the first stimulus element includes a force applicator that applies the peripheral stimulus, wherein the peripheral stimulus is a mechanical force having a force value to be applied to a first predefined area along the digit and wherein the second stimulus element includes a force applicator that applies the central stimulus, wherein the central stimulus is a mechanical force having a force value to be applied to a second predefined area along a surface of the patient.

37. The device of claim 36, further comprising:
a monitoring component operable to measure the force value of the mechanical force applied along the digit or the surface of the patient; and
an indicator in communication with the monitoring component operable to display the force value of the mechanical force applied along the digit or the surface of the patient.

38. The device of claim 37, wherein the monitoring component and indicator are electronic.

39. The device of claim 37, wherein the monitoring component and indicator are analog.

40. The device of claim 35, wherein the first stimulus element is an electrode having a first effective surface area that applies the peripheral stimulus to a first predefined area corresponding with the first effective surface area along the digit of the patient and wherein the second stimulus element is an electrode having a second effective surface area that applies the central stimulus to a second predefined area along a surface of the patient corresponding with the second effective surface area, wherein peripheral stimulus and central stimulus are each an electrical stimulus.

41. The device of claim 35, wherein the first stimulus element is a laser portal that applies the central stimulus to a first predefined area corresponding with a first diameter of the laser along a surface of the digit of the patient and the second stimulus element is a laser portal that applies the peripheral stimulus to a second predefined area corresponding with a second diameter of the laser along the surface of the patient, wherein the peripheral stimulus and central stimulus are each a thermal stimulus.
